(19) United States
(12) Patent Application Publication
(10) Pub. No.: US 2013/0287810 A1
(43) Pub. Date: Oct. 31, 2013

(54) VACCINE COMPOSITIONS AND USES THEREOF

(71) Applicants: Mansour Mohamadzadeh, Frederick, MD (US); Todd R. Klaenhammer, Raleigh, NC (US)

(72) Inventors: Mansour Mohamadzadeh, Frederick, MD (US); Todd R. Klaenhammer, Raleigh, NC (US)

(73) Assignee: NORTHWESTERN UNIVERSITY, Evanston, IL (US)

(21) Appl. No.: 13/745,253

(22) Filed: Jan. 18, 2013

Related U.S. Application Data
(63) Continuation of application No. 12/906,428, filed on Oct. 18, 2010, now abandoned.
(60) Provisional application No. 61/252,456, filed on Oct. 16, 2009.

(51) Int. Cl. A61K 39/145; A61K 39/07

(52) U.S. Cl. CPC A61K 39/145; A61K 39/07
USPC 424/192.1; 536/23.4; 435/252.3

(57) ABSTRACT

The present invention relates to vaccine compositions and uses thereof. Embodiments of the present invention provide oral bacterial (e.g., probiotic lactic acid bacteria) vaccine delivery systems comprising an antigen and a dendritic cell-targeting peptide. Such compositions target vaccines to dendritic cells, resulting in a high level of humoral and acquired immunity, including both mucosal and systemic immunity. Such delivery systems find use in the specific delivery of a wide variety of oral vaccines to subjects.

### CROSS-REFERENCE TO RELATED APPLICATION

This application is a continuation of U.S. patent application Ser. No. 12/906,428, filed Oct. 18, 2010, which claims priority to U.S. Provisional Patent Application No. 61/252,456, filed Oct. 16, 2010, each of which are herein incorporated by reference in its entirety.

### STATEMENT REGARDING FEDERALLY SPONSORED RESEARCH OR DEVELOPMENT

This invention was made with government support under Grant No. R21-AI059590 awarded by the National Institutes of Health. The government has certain rights in the invention.

### FIELD OF THE INVENTION

The present invention relates to vaccine compositions and uses thereof. Embodiments of the present invention provide oral bacterial (e.g., probiotic lactic acid bacteria) vaccine delivery systems comprising an antigen and a dendritic cell-targeting peptide. Such compositions target vaccines to dendritic cells, resulting in a high level of humoral and acquired immunity, including both mucosal and systemic immunity. Such delivery systems find use in the specific delivery of a wide variety of oral vaccines to subjects.

### BACKGROUND OF THE INVENTION

The use of vaccines against infectious microbes has been critical to the advancement of medicine. Vaccine strategies combined with, or without, adjuvants have been established to eradicate various bacterial and viral pathogens. There has been more than a 95% decline in morbidity and mortality with various childhood infections since the employment of vaccine technologies and their universal utilization. This is evidenced by the fact that there has been no smallpox cases reported in the world for more than three decades and, moreover, poliomyelitis has now been entirely abolished in Europe and North America. Thus, novel vaccine technologies and further refinement of existing methods and strategies attract talented scientists into the field. The establishment of mucosal vaccines, either for protection against microbes or for oral-tolerance immunotherapy, utilizes excellent antigen delivery and immune-modulatory adjuvants in vivo.

Additional vaccines that exhibit high efficacy and efficient delivery are needed in the art.

### SUMMARY OF THE INVENTION

The present invention relates to vaccine compositions, kits and uses thereof. Embodiments of the present invention provide oral bacterial (e.g., probiotic lactic acid bacteria) vaccine delivery systems comprising an antigen and a dendritic cell-targeting peptide. Such compositions target vaccines to dendritic cells, resulting in a high level of humoral and acquired immunity, including both mucosal and systemic immunity. Such delivery systems find use in the specific delivery of a wide variety of oral vaccines to subjects.

For example, in some embodiments, the present invention provides a composition comprising a nucleic acid encoding a fusion polypeptide comprising a dendritic cell-targeting peptide fused to an antigen of interest (e.g., an antigen derived from a pathogenic microorganism or a cancer cell) and optionally a pharmaceutically acceptable carrier. In some embodiments, the present invention provides a fusion polypeptide comprising a dendritic cell-targeting peptide fused to an antigen of interest. The present invention is not limited to a particular dendritic cell-targeting peptide. Any peptide that targets the fusion protein to a dendritic cell is suitable for use in the compositions and methods described herein. For example, in some embodiments, the dendritic cell-targeting peptide comprising or consists of the sequence FYPSYHSTPQRP (SEQ ID NO:1). The present invention is not limited to a particular antigen. One of skill in the art knows well how to identify antigens from exemplary pathogens (e.g., a viral antigen or a bacterial antigen). Examples include, but are not limited to B. anthrax, influenza and human immunodeficiency virus (HIV).

Further embodiments of the present invention provide a bacteria (e.g., a probiotic lactic acid bacteria) comprising the above described nucleic acid and polypeptide compositions. The present invention is not limited to a particular bacteria. Exemplary bacteria include, but are not limited to, Lactobacillus, Leuconostoc, Pediococcus, Lactococcus, or Streptococcus species (e.g., Lactobacillus gasseri or Lactobacillus acidophilus).

Embodiments of the present invention provide kits comprising the vaccines and bacteria described herein.

Additional embodiments of the present invention provide an immunization method, comprising administering a bacteria (e.g., a probiotic lactic acid bacteria) comprising a nucleic acid encoding a fusion polypeptide comprising a dendritic cell-targeting peptide fused to an antigen of interest to a subject, wherein administering confers immunity to the antigen to the subject. In some embodiments, the administration is oral or intranasal.

Additional embodiments are described herein.

### DESCRIPTION OF THE FIGURES

FIG. 1 shows plasmids for expression of rPA peptide fusions. Map of constitutive rPA/DC-peptide fusions and control plasmid, pTRK895 (A) and schematic of the expression cassettes for DCpep and Ctrlpep, pTRK896 and pTRK895 (B), respectively.

FIG. 2 shows Western blots of PA-DC and PA-Ctrl expressed proteins.

FIG. 3 shows protective immunity against B. anthracis Sterne. (A) Vaccination schedule. (B and C) Mouse survival.

FIG. 4 shows detection of Anti-PA antibodies. After the vaccination regime, sera were derived from each group of mice just before and after challenge with B. anthracis Sterne for assays of anti-PA antibodies (A) and the anti-toxin neutralizing antibodies (B).

FIG. 5 shows detection of IgA-expressing cells within the small intestine. (A) After isolation of jejunum and ileum, these tissues were fixed in 10% formalin and processed into paraffin blocks. (B) IgA⁺ cells of the lamina propria (LP) of villi and Peyer's patches (PP) were evaluated by a semiautomated quantitative image analysis system.

FIG. 6 shows induction of cytokines in vivo. (A and B) Cytokines released into the blood of mice that were bled before (A) and after (B) Sterne challenge were analyzed by using mouse inflammatory and Th1/Th2 cytometric bead array kits. (C) T cell stimulation.

FIG. 7 shows depiction of vaccination, infection and analysis of immune responses of mice to be infected with ×31 or PR8 Influenza A strains.

FIG. 8 shows characterization of DC-binding peptides. A-B. SDS-PAGE of binding proteins. C. Endocytosis activity of DCs that internalizes the DC-peptide.

FIGS. 9A-C shows induction of immune responses against anthrax infection.

FIG. 10 shows an exemplary markerless gene replacement strategy for insertion of PA-DCpep or Hc-DCpep into a targented region of a bacterial genome.

FIG. 11 shows expression of PA-DC-pep by Lactobacillus gasseri.

FIG. 12 shows induction of protective immunity against B. anthracis.

FIG. 13 shows (A) induction of anti-PA antibodies in mice. B. Cytokine analysis using cytometric bead assay.

FIG. 14A-B shows PA-dependent T cell stimulation.

### DEFINITIONS

To facilitate understanding of the invention, a number of terms are defined below.

As used herein, the term “dendritic cell-targeting peptide” refers to a peptide that interacts with dendritic cells. In some embodiments, dendritic cell-targeting peptides are fused to antigens in order to target the antigens to dendritic cells for processing. The present invention is not limited to a particular dendritic cell-targeting peptide. In some embodiments, the peptide is FYPSYHSTPQRP (SEQ ID NO:1).

Where “amino acid sequence” is recited herein to refer to an amino acid sequence of a naturally occurring protein molecule, “amino acid sequence” and like terms, such as “polypeptide” or “protein” are not meant to limit the amino acid sequence to the complete, native amino acid sequence associated with the recited protein molecule.

As used herein, the term “peptide” refers to a polymer of two or more amino acids joined via peptide bonds or modified peptide bonds. As used herein, the term “dipeptides” refers to a polymer of two amino acids joined via a peptide or modified peptide bond.

The term “wild-type” refers to a gene or gene product that has the characteristics of that gene or gene product when isolated from a naturally occurring source. A wild-type gene is that which is most frequently observed in a population and is thus arbitrarily designed the “normal” or “wild-type” form of the gene. In contrast, the terms “modified”, “mutant”, and “variant” refer to a gene or gene product that displays modifications in sequence and or functional properties (i.e., altered characteristics) when compared to the wild-type gene or gene product. It is noted that naturally-occurring mutants can be isolated; these are identified by the fact that they have altered characteristics when compared to the wild-type gene or gene product.

As used herein, the term “purified” or “to purify” refers to the removal of contaminants from a sample. For example, antigens are purified by removal of contaminating proteins. The removal of contaminants results in an increase in the percent of antigen (e.g., antigen of the present invention) in the sample.

The term “recombinant DNA molecule” as used herein refers to a DNA molecule that is comprised of segments of DNA joined together by means of molecular biological techniques.

The term “recombinant protein” or “recombinant polypeptide” as used herein refers to a protein molecule that is expressed from a recombinant DNA molecule.

The term “native protein” as used herein to indicate that a protein does not contain amino acid residues encoded by vector sequences; that is the native protein contains only those amino acids found in the protein as it occurs in nature. A native protein may be produced by recombinant means or may be isolated from a naturally occurring source.

As used herein the term “portion” when in reference to a protein (as in “a portion of a given protein”) refers to fragments of that protein. The fragments may range in size from four consecutive amino acid residues to the entire amino acid sequence minus one amino acid.

The term “Western blot” refers to the analysis of protein(s) (or polypeptides) immobilized onto a support such as nitrocellulose or a membrane. The proteins are run on acrylamide gels to separate the proteins, followed by transfer of the protein from the gel to a solid support, such as nitrocellulose or a nylon membrane. The immobilized proteins are then exposed to antibodies with reactivity against an antigen of interest. The binding of the antibodies may be detected by various methods, including the use of radiolabelled antibodies.

The term “antigenic determinant” as used herein refers to that portion of an antigen that makes contact with a particular antibody (i.e., an epitope). When a protein or fragment of a protein is used to immunize a host animal, numerous regions of the protein may induce the production of antibodies that bind specifically to a given region or three-dimensional structure on the protein; these regions or structures are referred to as antigenic determinants. An antigenic determinant may compete with the intact antigen (i.e., the “immunogen” used to elicit the immune response) for binding to an antibody.

As used herein, the term “immunogen” means a substance that induces a specific immune response in a host animal. The immunogen may comprise a whole organism, killed, attenuated or live; a subunit or portion of an organism; a recombinant vector containing an insert with immunogenic properties; a piece or fragment of DNA capable of inducing an immune response upon presentation to a host animal; a protein, a glycoprotein, a lipoprotein, a polypeptide, a peptide, an epitope, a hapten, or any combination thereof.

As used herein, the term “adjuvant” means a substance added to a vaccine to increase a vaccine's immunogenicity. Known vaccine adjuvants include, but are not limited to, oil and water emulsions (for example, complete Freund's adjuvant and incomplete Freund's adjuvant), in particular oil-in-water emulsions, water-in-oil emulsions, water-in-oil-in-water emulsions. They include also for example saponin, aluminum hydroxide, dextran sulfate, carbomer, sodium alginate, “AVRIDINE” (N,N-dioctadecyl-N′,N′-bis(2-hydroxyethyl)-propanediamine), paraffin oil, muramyl dipeptide, cationic lipids (e.g., DMRIE, DOPE and combinations thereof) and the like. In some embodiments, carrier bacteria of embodiments of the present invention serve as adjuvants.

As used herein, the terms “pharmaceutically acceptable carrier” and “pharmaceutically acceptable vehicle” are interchangeable and refer to a fluid vehicle for containing vaccine immunogens that can be administered to a host without significant adverse effects.

As used herein, the term “vaccine composition” includes at least one antigen or immunogen in a pharmaceutically acceptable vehicle useful for inducing an immune response in a host. Vaccine compositions can be administered in dosages and by techniques well known to those skilled in the medical or veterinary arts, taking into consideration such factors as the age, sex, weight, species and condition of the recipient animal, and the route of administration.

As used herein, the term “host cell” refers to any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic animal.

The term “test compound” refers to any chemical entity, pharmaceutical, drug, and the like that can be used to treat or prevent a disease, illness, sickness, or disorder of bodily function, or otherwise alter the physiological or cellular status of a sample. Test compounds comprise both known and potential therapeutic compounds. A test compound can be determined to be therapeutic by screening using the screening methods of the present invention. A “known therapeutic compound” refers to a therapeutic compound that has been shown (e.g., through animal trials or prior experience with administration to humans) to be effective in such treatment or prevention.

The term “sample” as used herein is used in its broadest sense. As used herein, the term “sample” is used in its broadest sense. In one sense it can refer to a tissue sample. In another sense, it is meant to include a specimen or culture obtained from any source, as well as biological. Biological samples may be obtained from animals (including humans) and encompass fluids, solids, tissues, and gases. Biological samples include, but are not limited to blood products, such as plasma, serum and the like. These examples are not to be construed as limiting the sample types applicable to the present invention. A sample suspected of containing a human chromosome or sequences associated with a human chromosome may comprise a cell, chromosomes isolated from a cell (e.g., a spread of metaphase chromosomes), genomic DNA (in solution or bound to a solid support such as for Southern blot analysis), RNA (in solution or bound to a solid support such as for Northern blot analysis), cDNA (in solution or bound to a solid support) and the like. A sample suspected of containing a protein may comprise a cell, a portion of a tissue, an extract containing one or more proteins and the like.

### DETAILED DESCRIPTION OF THE INVENTION

The present invention relates to vaccine compositions and uses thereof. Embodiments of the present invention provide oral bacterial (e.g., probiotic lactic acid bacteria) vaccine delivery systems comprising an antigen and a dendritic cell-targeting peptide. Such compositions target vaccines to dendritic cells, resulting in a high level of humoral and acquired immunity, including both mucosal and systemic immunity. Such delivery systems find use in the specific delivery of a wide variety of oral vaccines to subjects.

The next generation of oral vaccines is ideally administered in a single, tolerable, efficacious dose that induces a robust neutralizing humoral and acquired immunity against specific microbial pathogens. Moreover, such vaccines are preferably safe, inexpensive, and stable. Ideally, vaccine delivery vectors stimulate immune responses at sites where pathogens interact with mammalian hosts, thereby generating the first eminent barriers against infection. An additional advantage of oral vaccination not usually observed with s.c. or intramuscular injection is the simultaneous induction of both mucosal and systemic immunity against the antigen of interest.

The mucosa represents the site for the first dynamic interactions between microbes and the human host. Accordingly, a robust and highly specialized innate, as well as adaptive, mucosal immune system protects the mucosal membrane from pathogens (Acheson et al., D W, Luccioli S (2004) Best Pract Res Clin Gastroenterol 18:387-404; Niedergang et al., (2005) Trends Microbiol 13:485-490). Although the mucosal site normally tolerates associated commensal microbiota, specific immunity is constantly induced against invading pathogens in mucosa-associated lymphoid tissues (MALT) through the homing specificity of activated effector lymphocytes (Holmgren J, et al. (2005) Immunol Lett 97:181-188; Holmgren et al., (2005) Nat Med 11:S45-S53).

Live attenuated vaccine vectors such as Samonella, Bortedella, and Listeria have been successfully used to deliver heterologous antigens (Roberts et al., (2000) Infect Immun 68:6041-6043; Stevenson et al., (2003) FEMS Immunol Med Microbiol 37:121-128; Saklani-Jusforgues et al., (2003) Infect Immun 71:1083-1090.). Although many of the properties related to their pathogenicity make them attractive candidates for inducing immune responses, the potential for reversion of attenuated strains to virulence is a significant safety concern. Moreover, these bacteria are highly immunogenic, which may prevent their use in vaccine regimens requiring multiple doses (Pouwels et al (1998) Int J Food Microbiol 41:155-167).

Accordingly, in some embodiments, the present invention provides vectors for oral delivery of vaccines that overcome the limitations of prior vaccines. Exemplary compositions and methods of their use are described below.

### I. Vectors for Delivery of Oral Vaccines

As described above, embodiments of the present invention provide oral bacterial vaccine delivery systems comprising a bacterial delivery vehicle that comprises a nucleic acid encoding an antigen—dendritic cell-targeting peptide fusion.

Additional exemplary information regarding oral dendritic cell-targeting vaccines is described, for example, in Mohamadzadeh, Cancer HIV Research, 2010 8:323; Tournier and Mohamadzadeh, Trends in Molecular Medicine, 2010, 16:303; Mohamadzadeh et al., Expert Vaccines 2008, 7:163; each of which is herein incorporated by reference in its entirety.

A. Bacterial Delivery System

The present invention is not limited to a particular bacterial delivery system. In some embodiments, probiotic lactic acid bacteria are utilized as delivery vectors for oral vaccines. Probiotics are defined as “live microorganisms that when administered properly, confer a health benefit to the host” (Ouwehand et al., (2002) Antonie Van Leeuwenhoek 82:279-289). Lactic acid bacteria (LAB) comprise a group of Gram-positive bacteria that include, for example, species of Lactobacillus, Leuconostoc, Pediococcus, Lactococcus, Streptococcus, as well as the more peripheral Aerococcus, Carnobacterium, Enterococcus, Oenococcus, Sporolactobacillus, Teragenococcus, Vagococcus, and Weisella. Lactobacillus species play a critical role as commensals in the gastrointestinal (GI) tract. Their ability to survive transit through the stomach, close association with the intestinal epithelium, immunomodulatory properties, and their safe consumption in large amounts make lactobacilli suitable as vaccine delivery vehicles. In some embodiments, enhancement of epitope bioavailability conferred by the delivery vehicle, specific species can be selected (Wells et al., supra).

In some embodiments, the vaccine carrier is, for example, a Lactobacillus species such as Lactobacillus acidophilus, Lactobacillus gasseri, Lactobacillus plantarum, Lactobacillus delbreuckii, Lactobacillus rhamnosus, Lactobacillus salivarius and Lactobacillus paracasei; and the heterofermentative species, Lactobacillus reuteri or Lactobacillus fermentum.

The present invention is not limited to the bacteria or lactic acid bacteria disclosed herein. One skilled in the art recognizes that other suitable species of bacteria may be utilized.

In some embodiments, bacteria comprise a nucleic acid encoding the dendritic cell-targeting-antigen fusion protein described herein. In some embodiments, the nucleic acid is on a self sustaining or replicating vector (e.g., a plasmid). In other embodiments, it is integrated into the bacterial chromosome. Methods for generating such bacteria are known in the art and are described, for example, in the Experimental section below.

B. Dendritic Cell-Targeting Peptides

Embodiments of the present invention provide dendritic cell targeting proteins that target fusion proteins to dendritic cells. Dendritic cells (DCs) are immune cells that form part of the mammalian immune system. Their main function is to process antigen material and present it on the surface to other cells of the immune system, thus functioning as antigen-presenting cells. They act as messengers between the innate and adaptive immunity.

Professional antigen presenting DCs have been identified in numerous tissue compartments, including the lamina propria (LP), the subepithelium, a T cell-rich zone of lymphoid tissue associated with the mucosa, and draining lymph nodes (Rescigno (2008) J Pediatr Gastroenterol Nutr 46:Suppl 1:E17-E19; Rescigno et al., (2008) Eur J Immunol 38:1483-1486). DCs located in or beneath the epithelium can sample and capture various bacterial antigens that cross the epithelial layer through M cells (Kelsall et al., (1996) Ann NY Acad Sci 778:47-54; Kelsall et al., (1996) J Exp Med 183:237-247; Niess et al., (2005) Science 307:254-258; Niess et al., (2005) Curr Opin Gastroenterol 21:687-691). Additionally, DCs within the LP, recruited by chemokines released by epithelial cells, reach the gut epithelia expressing occludin and claudin-1 molecules. These latter molecules facilitate penetration of these cells into the tight junctions between epithelial cells. DCs subsequently extend their probing dendrites into the lumen to sample commensal or microbial immunogens (Pamer, supra, Rescigno (2001) Nat Immunol 2:361-367; Kraehenbuhl et al., (2004) Science 303:1624-1625; Macpherson et al., (2004) Science 303:1662-1665). These cells then migrate into the lymphoid follicles wherein processed antigens are presented to B and T cells to initiate humoral (IgA) and T cell immune responses (Mohamadzadeh M, et al (2005), supra).

Accordingly, embodiments of the present invention provide vaccine compositions comprising a peptide that targets dendritic cells (e.g., “dendritic cell-targeting peptide”) fused to an antigen (e.g., an antigen from a pathogenic microorganism). The present invention is not limited to a particular dendritic cell-targeting peptide. Any peptide that targets the antigen of interest to a dendritic cell for processing is suitable for use in the compositions and methods described herein. For example, in some embodiments, the peptide is FYPSYHSTPQRP (SEQ ID NO:1). In other embodiments, one or more amino acid changes are incorporated into the targeting peptide of SEQ ID NO:1 (e.g., to generate variants of SEQ ID NO:1).

Variants of SEQ ID NO:1 may have “conservative” amino acid changes, wherein a substituted amino acid has similar structural or chemical properties. In some embodiments, a conservative amino acid substitution refers to the interchangeability of residues having similar side chains. For example, the amino acids glycine, alanine, valine, leucine, and isoleucine have aliphatic side chains; the amino acids serine and threonine have aliphatic-hydroxyl side chains; the amino acids asparagine and glutamine have amide-containing side chains; the amino acids phenylalanine, tyrosine, and tryptophan have aromatic side chains; the amino acids lysine, arginine, and histidine have basic side chains; and the amino acids cysteine and methionine have sulfur-containing side chains. In some embodiments, conservative amino acids substitution groups include, but are not limited to: valine-leucine-isoleucine, phenylalanine-tyrosine, lysine-arginine, alanine-valine, and asparagine-glutamine. In some embodiments, conservative amino acid substitutions may include the substitution of: alanine with serine; arginine with glutamine, histidine, or lysine; asperigine with glutamic acid, glutamine, lysine, histidine, or aspartic acid; aspartic acid with asperigine, glutamic acid, or glutamine; cysteine with serine or alanine; glutamine with asperigine, glutamic acid, lysine, histidine, aspartic acid, or arginine; glutamic acid with glycine, asperigine, glutamine, lysine, or aspartic acid; glycine with proline; histidine with asperigine, lysine, glutamine, arginine, or tyrosine; isoleucine with leucine, methionine, valine, or phenylalanine; leucine with isoleucine, methionine, valine, or phenylalanine; lysine with asperigine, glutamic acid, glutamine, histidine, or arginine; methionine with isoleucine, leucine, valine, or phenylalanine; phenylalanine with tryptophan, tyrosine, methionine, isoleucine, or leucine; serine with threonine or alanine; threonine with serine or alanine; tryptophan with phenylalanine or tyrosine; tyrosine with histidine, phenylalanine, or tryptophan; and/or valine with methionine, isoleucine, or leucine.

Variants can be screened for activity using methods known in the art (e.g., the examples described in the Experimental section below) to determine peptides with dendritic cell-targeting activity. Exemplary variants are shown in Table 3 below.

TABLE 3
Dendritic Cell Targeting Peptides

| | |
|---|---|
| SEQ ID NO: 1 | FYPSYHSTPQRP |
| SEQ ID NO: 50 | NYPSYHSTPQRP |
| SEQ ID NO: 51 | FNPSYHSTPQRP |
| SEQ ID NO: 52 | FYNSYHSTPQRP |
| SEQ ID NO: 53 | FYPNYHSTPQRP |
| SEQ ID NO: 54 | FYPSNHSTPQRP |
| SEQ ID NO: 55 | FYPSYNSTPQRP |
| SEQ ID NO: 56 | FYPSYHNTPQRP |
| SEQ ID NO: 57 | FYPSYHSNPQRP |
| SEQ ID NO: 58 | FYPSYHSTNQRP |
| SEQ ID NO: 59 | FYPSYHSTPNRP |
| SEQ ID NO: 60 | FYPSYHSTPQNP |
| SEQ ID NO: 61 | FYPSYHSTPQRN |

“N” is any amino acid. Preferably, “N” is a conservative amino acid change as compared to SEQ ID NO: 1.

The present invention is not limited to a particular antigen. In some embodiments, antigens are derived from a pathogenic microorganism (e.g., a virus or a bacteria). Embodiments of the present invention are illustrated with B. anthracis, influenza virus (e.g., H1N1), and HIV. In some embodiments, antigens are derived from cancer cells (e.g., to generate cancer vaccines). One skilled in the art recognizes that these are exemplary, non-limiting examples of antigens that can be utilized in embodiments of the present invention.

Embodiments of the present invention provide kits and pharmaceutical compositions comprising the bacterial cells and dendritic cell-targeting peptide fusions described herein. In some embodiments, pharmaceutical compositions comprise a bacteria comprising a nucleic acid (e.g., on a plasmid or integrated into the chromosome) encoding a dendritic cell-targeting peptide-antigen fusion protein, along with a pharmaceutical carrier.

In some embodiments, kits comprise a bacteria comprising a nucleic acid (e.g., on a plasmid or integrated into the chromosome) encoding a dendritic cell-targeting peptide-antigen fusion protein alone with any other components necessary, sufficient or useful for research, clinical, or screening applications. For example, in some embodiments, kits for use in vaccination comprise devices for administering the vaccine (e.g., syringes or other vehicles for oral and nasal administration), temperature control components (e.g., refrigeration or other cooling components), sanitation components (e.g., alcohol swabs for sanitizing the site of administration) and instructions for administering the vaccine.

### II. Vaccination Methods

Embodiments of the present invention provide vaccines comprising a bacteria comprising a nucleic acid (e.g., on a plasmid or integrated into the chromosome) encoding a dendritic cell-targeting peptide-antigen fusion protein, along with a pharmaceutical carrier.

The present vaccine may be administered, for example, orally, nasally, or parenterally. Examples of parenteral routes of administration include intradermal, intramuscular, intravenous, intraperitoneal, subcutaneous and intranasal routes of administration. In some preferred embodiments, the vaccine is administered orally or intranasally.

In some embodiments, oral and nasal administration routes have the advantage of specific activation of DCs, directional elicitation of humoral and T-cell-mediated immunity by these cells, and a delivery system that can serve as a safe and potent adjuvant. When administered orally, vaccines of embodiments of the present invention flood the GI tract where, during transit, they secrete immunogenic fusion proteins into the intestinal lumen that specifically binds to its ligand expressed on mucosal DCs via DC-binding moieties. In the case of nonsecreted proteins, lactobacilli expressing immunogenic fusion protein are taken up by M cells and transported to gut DCs wherein immunogenic fusion proteins can be captured, processed and presented to T cells, inducing antigen-specific T-cell immune responses.

In some embodiments, vaccines are administered in a single or multiple doses to an individual in need. In some embodiments, booster or additional doses are administered as needed.

When administered as a solution, the present vaccine may be prepared in the form of an aqueous solution, a syrup, an elixir, or a tincture. Such formulations are known in the art, and are prepared by dissolution of the antigen and other appropriate additives in the appropriate solvent systems. Such solvents include water, saline, ethanol, ethylene glycol, glycerol, Al fluid, etc. Suitable additives known in the art include certified dyes, flavors, sweeteners, and antimicrobial preservatives, such as thimerosal (sodium ethylmercurithiosalicylate). Such solutions may be stabilized, for example, by addition of partially hydrolyzed gelatin, sorbitol, or cell culture medium, and may be buffered by methods known in the art, using reagents known in the art, such as sodium hydrogen phosphate, sodium dihydrogen phosphate, potassium hydrogen phosphate and/or potassium dihydrogen phosphate.

Liquid formulations may also include suspensions and emulsions. The preparation of suspensions, for example using a colloid mill, and emulsions, for example using a homogenizer, is known in the art.

Parenteral dosage forms, designed for injection into body fluid systems, utilize proper isotonicity and pH buffering to the corresponding levels of body fluids. Parenteral formulations are generally sterilized prior to use.

Isotonicity can be adjusted with sodium chloride and other salts as needed. Other solvents, such as ethanol or propylene glycol, can be used to increase solubility of ingredients of the composition and stability of the solution. Further additives which can be used in the present formulation include dextrose, conventional antioxidants and conventional chelating agents, such as ethylenediamine tetraacetic acid (EDTA).

### EXPERIMENTAL

The following examples are provided in order to demonstrate and further illustrate certain preferred embodiments and aspects of the present invention and are not to be construed as limiting the scope thereof.

### Example 1

### Dendritic Cell Targeting of Bacillus anthracia Protective Antigen Expressed by Lactobacillus acidophilus protects mice from lethal challenge

### Materials and Methods

### Animals.

A/J mice (age 6-8 weeks) were purchased from the National Cancer Institute (NCl) in Frederick, Md. Mice were housed in clean standard conditions in the animal care facility at the US Army Medical Research Institute of Infectious Diseases (USAMRIID). Research was conducted in compliance with the Animal Welfare Act and other federal statutes and regulations related to animals and experiments involving animals. The principles stated in the Guide for the Care and Use of Laboratory Animals were followed. The research was conducted at USAMRIID, which is fully accredited by the Association for Assessment and Accreditation of Laboratory Animal Care International.

Expression of Recombinant B. anthracis PA-DC Peptide Fusion by L. acidophilus.

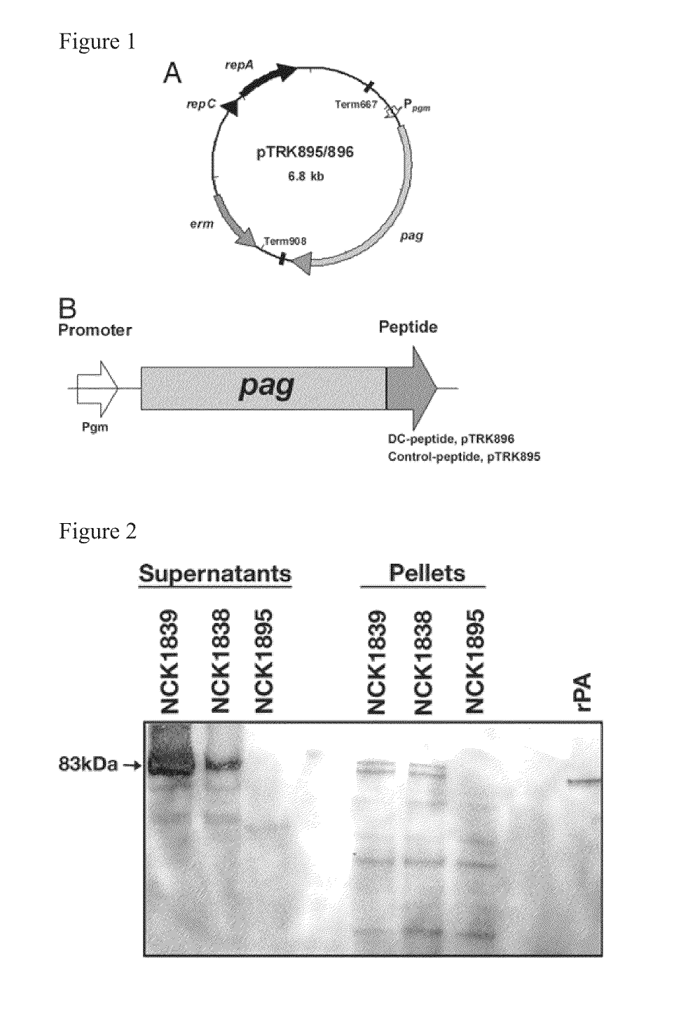

To express PA of B. anthracis (Ivins et al., (1986) Infect Immun 54:537-542) fused to DC peptide or the control peptide, 2 constructs were made. Each peptide encodes a PA C-terminal fusion to either a DC targeting peptide (FYPSYHSTPQRP; SEQ ID NO:1) or a control peptide (EPIHPETTFTNN; SEQ ID NO:2) designated pPAGDC and pPAGctrl, respectively (FIG. 1A). Subsequently, the recombinant PA (rPA) fusion genes were PCR-cloned into expression vector pTRK882, a shuttle vector based on the strong constitutive pgm promoter of L. acidophilus. Plasmids pPAGctrl and pPAGDC containing the PA-control peptide or PA-DC peptide fusions were first constructed. Primers PA-F(5′-ATGCGGATCCCAAAAAGGAGAACGTATATG-3′; SEQ ID NO:3) and PA-R (5′-GCAATTAACCCTCACTAAAG-3′; SEQ ID NO:4) were used to amplify the rPA fusion genes for cloning into pTRK882. rPA fusion genes were cloned into the BamHI and NotI sites of pTRK882 yielding pTRK895 and pTRK896. Subsequently, the constructed plasmids were transformed into L. acidophilus NCFM by electroporation (Table 1). Transformants were initially selected by ERM (Sigma) resistance and then screened by plasmid isolation, followed by restriction digestion (FIGS. 1A and B). The plasmids were additionally verified by nucleotide sequencing of the junction points between the vector and inserted DNA.

### Western Blot Analyses.

To examine for rPA expression by L. acidophilus, cultures NCK1838 (PA-Control peptide), NCK1839 (PA-DCpeptide), and NCK1895 (empty vector) were grown to midlog phase (O.D.=0.4-0.6) in MRS broth (Difco) supplemented with ERM, centrifuged, and the cell pellets and supernatants were collected for SDS/PAGE. Cultures of the constitutively expression constructs NCK1838, NCK1839, and NCK1895 were grown to midlog phase in MRS supplemented with ERM (5 μg/mL). Cell pellets were then lysed by bead-beating. Proteins from supernatants were precipitated by using trichloroacetic acid (TCA) and pelleted by centrifugation. The total protein (10 μg) from both supernatants and cell pellets were loaded onto a SDS/PAGE gel. rPA was used as a positive control and NCK1895 containing the empty vector pTRK882 served as a negative control, respectively. After electrophoresis, the proteins were transferred to a nitrocellulose membrane and probed with anti-PA antibody conjugated with HRP. Blots were then washed, treated with 3,3′,5,5′-tetramethyl benzidine (TMB) substrate (KPL), and visualized by a Phosphorlmager.

### Mouse DC Culture.

Mouse DCs were generated as previously described (Pulendran (2004) Eur J Immunol 34:66-73). Briefly, after removing bone marrow cells from mouse femurs, the cells were washed and cultured in complete RPMI medium 1640 plus 10% FCS and 25 ng/mL mouse recombinant GM-CSF at 37° C. The phenotype of these DCs on day 8 was determined by a FACS Cantoll flow cytometer (BD). Mouse DCs were positive for CD11c, CD11b, and MHC II. Subsequently, the endocytotic activity was determined by incubating mouse DCs with Alexa Fluor 647 (Invitrogen)-labeled L. acidophilus strains including NCK1895, NCK1838, and NCK1839 at a ratio of 1:10 for 1 h at 37° C. As a control, a portion of DCs were incubated with Alexa Fluor 647-labeled L. acidophilus strains on ice. These cells were then washed with cold PBS/0.1% FCS and analyzed by flow cytometry (Mohamadzadeh (2001) J Exp Med 194:1013-1020).

### In Vivo Vaccination.

L. acidophilus strains expressing PA-DCpep, PA-Ctrlpep, and a null vector control were grown at 37° C. in MRS broth supplemented with ERM (5 μg/mL) for 72 h in tightly capped flasks without shaking Cells were centrifuged and washed twice in PBS before a final resuspension at 10⁹ CFU/250 μL in PBS. Subsequently, groups of mice were orally vaccinated with L. acidophilus NCK 1839 (PA-DCpep), L. acidophilus NCK1838 (PA-Ctrlpep), and L. acidophilus NCK 1895 (empty vector) by gavage of 250 μL containing ≈10⁹ CFU. Vaccination was repeated 3 times on a weekly basis. Two weeks later, the groups of mice were boosted twice. Seven days after the final boost, the mice were i.p. challenged with B. anthracis Sterne pXO1⁺/pXO2⁻ (5×10⁴ CFU per mouse) (Welkos et al., (1986) Infect Immun 51:795-800). Survival was monitored until day 40. Additionally, blood was taken from each mouse before and after challenge to determine the levels of anti-PA antibodies, PA-neutralizing antibodies, and cytokines released into the peripheral blood.

### Anti-PA Antibody Analysis.

The anti-PA antibody response was measured by ELISA (Albrecht (2007), supra; Zegers (1999) J Appl Microbiol 87:309-314). Briefly, microtiter plates were coated with rPA overnight at 4° C. Plates were then blocked with milk (6%) in PBS. Subsequently, mouse sera were added to wells in 2 log serial dilutions (1:40 to 1:81920) and the plates incubated for 2 h at 37° C. Plates were washed, and serum antibodies bound to rPA were detected by adding HRP-conjugated goat anti-mouse IgG (BD Biosciences). Plates were then incubated for 1 h at 37° C. 3,3′,5,5′-Tetramethylbenzidine (TMB) substrate was added and incubated at 37° C. for 5-10 min. Absorbencies were determined at 405 nm after neutralization with 50 μL of hydrochloric acid (1 M).

B. anthracis Toxin-Neutralizing Antibodies.

To determine the levels of neutralizing anti-PA antibodies elicited by L. acidophilus expressing PA-DCpep versus its controls, a toxin-neutralization assay was used (Albrecht (2007), supra). This assay was performed to demonstrate that anti-PA antibodies released by peripheral blood immune cells were capable of preventing the association of PA to B. anthracis lethal factor (LF) or the binding of lethal toxin to cell receptors, thereby resulting in increased survival of B. anthracis lethal toxin-treated macrophages. Briefly, serially diluted mouse sera were incubated at 37° C. with B. anthracis lethal toxin (PA 100 ng/mL and LF 20 ng/mL). After 1 h, the mixture was added to J774A.1 macrophages (10⁵ per well) in a 96-well plate. After 4 h incubation at 37° C., 25 μL of MTT (1 mg/mL) dye was added and the cells were further incubated for 2 h. The reaction was stopped by adding an equal volume of lysis buffer [50% DMF and 20% SDS (pH 7.4)]. Plates were incubated overnight at 4° C., and the absorbance was read at 570 nm in a multiwell plate reader.

### Detection of IgA.

The level of IgA was determined within the small intestine. Briefly, for immunohistological studies, the jejunum and ileum were isolated from mice in each vaccinated group (2 mice per group) for staining of IgA-expressing cells. Subsequently, the tissues were fixed in 10% formalin and processed into paraffin blocks. Serial tissue sections (5-μm thick) were mounted on glass slides and IgA expressing cells were visualized with a rabbit anti-mouse IgA polyclonal antibodies (Zymed Laboratories) and a secondary goat anti-rabbit HRP antibody (DAKO). IgA⁺ regions of the small intestine, including the LP of villi and PP were evaluated by a semiautomated quantitative image analysis system of the immunohistochemically labeled tissues (ACIS II; DakoCytomation. From digitized images of the stained tissue sections, the percentage of pixels in each tissue that contained the immunostain chromogen was measured and expressed as a percentage of the scanned area (positive pixels/positive+negative pixels). The mean intensity of each chromogen-containing pixel was calculated and expressed as the mean pixel intensity.

### Cytokine Analysis.

Cytokines released into the peripheral blood of mice that were bled by tail nick before and after B. anthracis Sterne challenge were analyzed by using mouse inflammatory and Th1/Th2 cytometric bead array kits (BD Biosciences). Briefly, the bead mixture (50 μL) was combined with the mouse sera (50 μL, vol/vol), or standards (50 μL), and phycoerythrin (50 μL). Subsequently, samples were incubated for 2 h at room temperature in the dark. These samples were washed, centrifuged, resuspended in wash buffer (300 μL) and then analyzed by a FACS Cantoll flow cytometer (BD). Analysis software (BD CellQuest) allowed for calculation of cytokine values in sera at picogram-per-milliliter amounts.

### T Cell Stimulation.

Highly purified, bone marrow-derived DCs were prepared as described above. The rPA-treated and untreated, DCs (10⁴ per well) were seeded in round-bottomed microtiter plates and subsequently cultured for 12 h at 37° C. T cells (10⁵ per well) from mice that survived the B. anthracis Sterne challenge were isolated from mesenteric lymph nodes by using a negative magnetic bead method. These cells were then cocultured with PA-treated or -untreated DCs for 5 days. Afterward, cell supernatants were harvested and cytokine release analyzed by using CBA mouse TH1/TH2 kits on the FACS Cantoll flow cytometer (BD).

TABLE 1
Bacterial strains and plasmids

| Strain or plasmid | Relevant characteristics |
|---|---|
| L. acidophilus | |
| NCFM | Human intestinal isolate |
| NCK 1838 | NCFM w/pTRK895 (PA-Ctrlpep) |
| NCK 1839 | NCFM w/pTRK896 (PA-DCpep) |
| NCK 1895 | NCFM w/pTRK882 (empty vector) |
| Escherichia coli | |
| MC-1061 | Str^{r}, E. coli transformation host |
| Plasmids | |
| pTRK882 | 4.5-kb, Em^{r}, constitutive expression vector, |
| | P_{pgm} promoter |
| pPAGctrl | 5.7-kb, Amp^{R} source of pagctrl gene |
| pPAGDC | 5.7-kb, Amp^{R} source of pagDC gene, |
| | encodes PADC |
| pTRK895 | 6.8-kb, Erm^{r}, pTRK882::pagcon, |
| | constitutive expression of PA-ctrlpep |
| pTRK896 | 6.8-kb, Erm^{r}, pTRK882::pagdc, |
| | constitutive expression of PA-DCpep |

### Results

Expression of rPA in L. acidophilus.

To establish a platform for oral vaccine delivery, the constructed plasmids were successfully transformed into L. acidophilus (FIGS. 1A and B and Table 1). The PA fusion proteins were secreted by L. acidophilus and were identified in the cell supernatants. FIG. 2 shows the lanes of an SDS/PAGE gel in which supernatants or cell pellets of cultures of L. acidophilus NCK1838 (PA-Ctrlpep), NCK1839 (PA-DCpep), and NCK1835 (empty vector) were loaded and subjected to Western blot analysis using anti-PA antibody. The identity of the 2 83-kDa bands in the culture supernatants were confirmed as the PA fusion proteins (FIG. 2).

L. acidophilus Interactions with DCs.

To demonstrate that L. acidophilus strains expressing PA fusions and their controls can be captured by mouse DCs, Alexa Fluor 647-labeled bacteria were cocultured with DCs. Data show that mouse DCs efficiently captured labeled L. acidophilus strains, indicating that the endocytotic pathway of DCs was not impaired. Additionally, the cytokines released by DCs treated with these L. acidophilus strains were studied. Low levels of IL-12 production was detected in DCs treated with L. acidophilus NCK 1839 expressing PA-DCpep. Other cytokines such as TNFα, IL-6, and IL-10 were induced at approximately the same levels in mouse DCs treated with all 3 recombinant L. acidophilus strains.

Vaccination with Recombinant L. acidophilus.

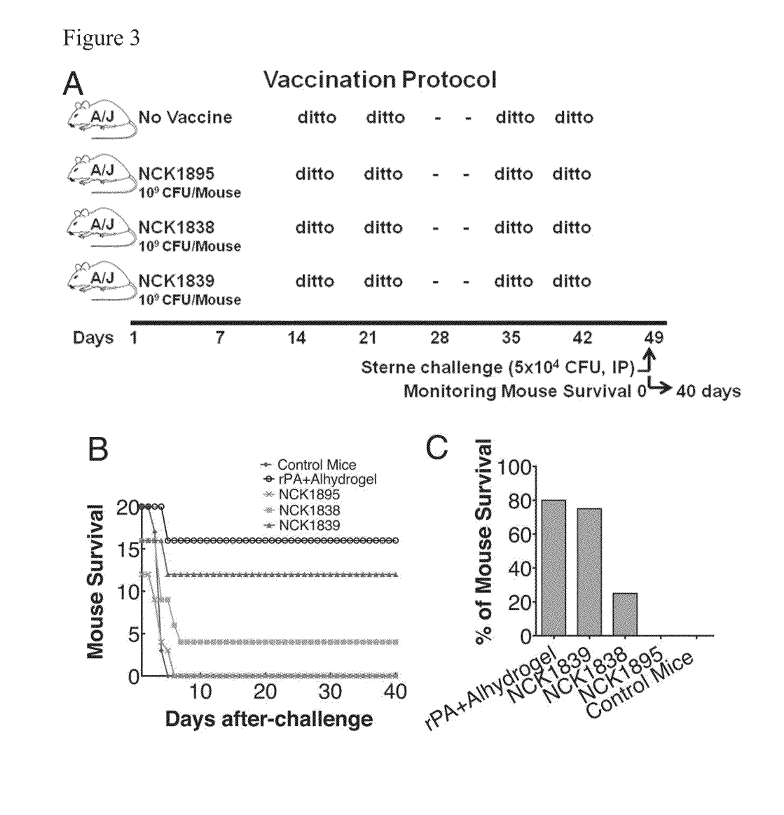

L. acidophilus strains expressing PA-Dcpep or PA-Ctrlpep or harboring the vector were grown to late log phase in deMan, Rogosa, and Sharpe (MRS) medium with erythromycin (ERM) and then pelleted, washed, and resuspended at 10⁹ CFU in 250 μL, in PBS. A/J mice were orally vaccinated with L. acidophilus NCK1839 (PA-DCpep), L. acidophilus NCK1838 (PA-Ctrlpep), or L. acidophilus NCK1895 (empty vector), and challenged with B. anthracis Sterne (5×10⁴ CFU per mouse). The results showed that 12 of 16 mice (75%) vaccinated with L. acidophilus expressing PA-DCpep survived, whereas only 4 of 16 mice in the control group vaccinated with L. acidophilus expressing PA-Ctrlpep survived the lethal challenge with B. anthracis Sterne (FIG. 3 A-C). All other groups, including L. acidophilus containing null vector (n=16) or PBS alone (n=20), succumbed to the lethal challenge (FIGS. 3 B and C). The current anthrax vaccine, rPA adsorbed to alhydrogel, given in a single s.c. injection, protected 16 of 20 mice from B. anthracis Sterne lethal challenge (FIGS. 3 B and C). Thus, results from these studies further demonstrate the efficacy of employing probiotic lactic acid bacteria in vaccine platforms, whereupon microbial immunogens such as B. anthracis PA can be delivered by using small DC-targeting peptides fused to the C terminus of the antigen.

### Anti-PA Antibody Analysis.

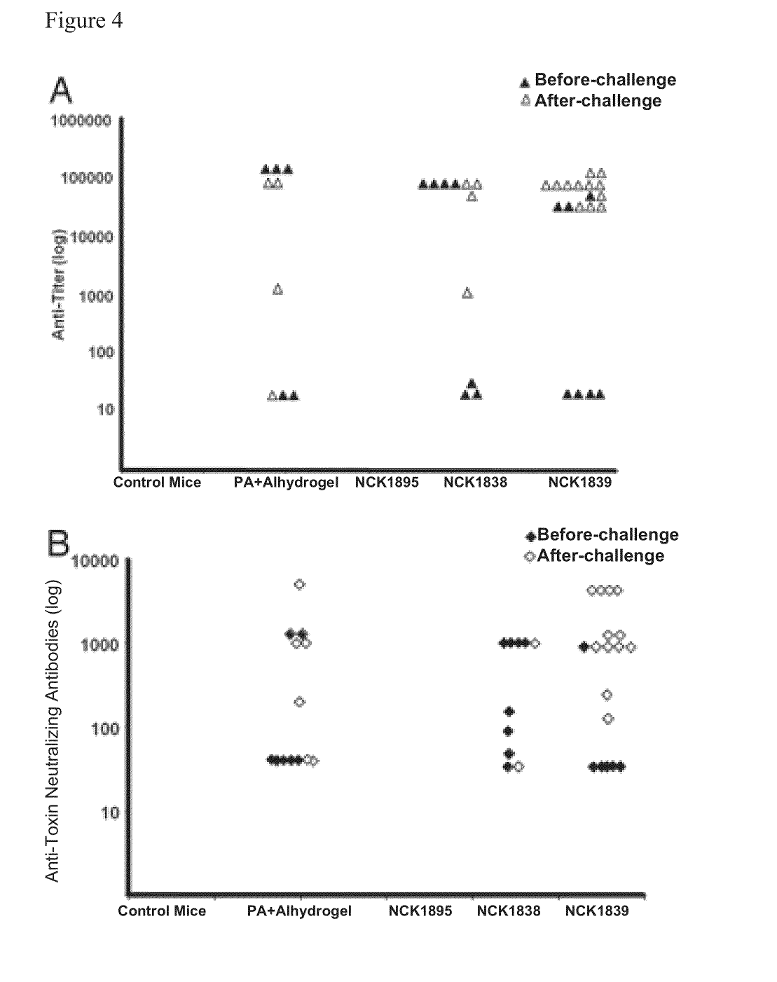

The production of anti-PA antibodies in vaccinated mice as well as in those mice that survived the challenge was analyzed by ELISA. Sera derived from the mice that survived challenge contained high titers of anti-PA antibodies, which were comparable with antibody levels from mice in the group vaccinated with rPA plus alhydrogel (FIG. 4A). Mice vaccinated with L. acidophilus expressing PA-Ctrlpep also showed a range of anti-PA titers, but the titers were not sufficient to elicit the same degree of protective immunity to allow their survival.

B. anthracis Toxin-Neutralizing Antibodies.

To determine the levels of B. anthracis toxin-neutralizing anti-PA antibodies elicited by L. acidophilus expressing PA-DCpep versus its control, a toxin-neutralization assay was performed (Albrecht M T, et al. (2007) Infect Immun 75:5425-5433). This assay was performed to demonstrate that anti-PA antibodies released by peripheral immune cells were capable of preventing the association of PA to the B. anthracis lethal factor (LF) or the binding of lethal toxin (PA+LF) to cell receptors, thereby resulting in increased survival of B. anthracis lethal toxin-treated macrophages. Data show that toxin-neutralizing antibody titers were reported as the reciprocal of the dilution that showed ≧30% cellular protection. These results demonstrate that L. acidophilus expressing PA-DCpep elicited high levels of neutralizing anti-PA antibodies in vivo that may have been a factor in the protection of those mice against Sterne challenge (FIG. 4B).

Detection of IgA⁺ Cells within Small Intestine.

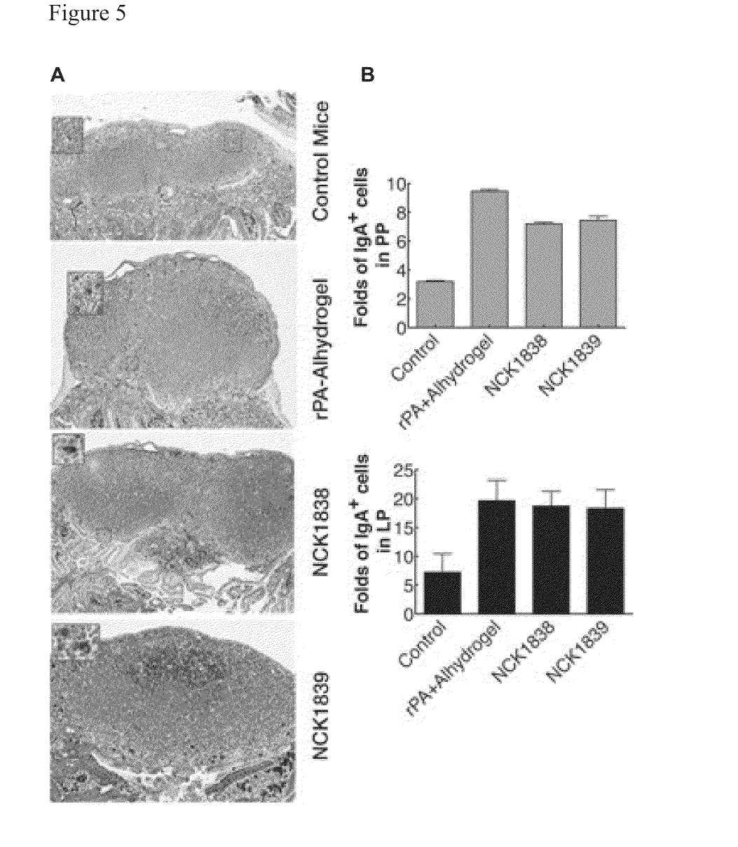

Immunostaining data of small intestinal sections showed higher expression of IgA⁺ plasma cells in the LP of villi and PP and occasional cells transmigrating the epithelium from all groups of mice compared with unvaccinated mice (FIGS. 5 A and B). Additionally, there was extracellular labeling of secreted IgA in these areas that was especially prominent along the apical surface of some epithelial cells (FIG. 5A).

### Induction of Cytokines.

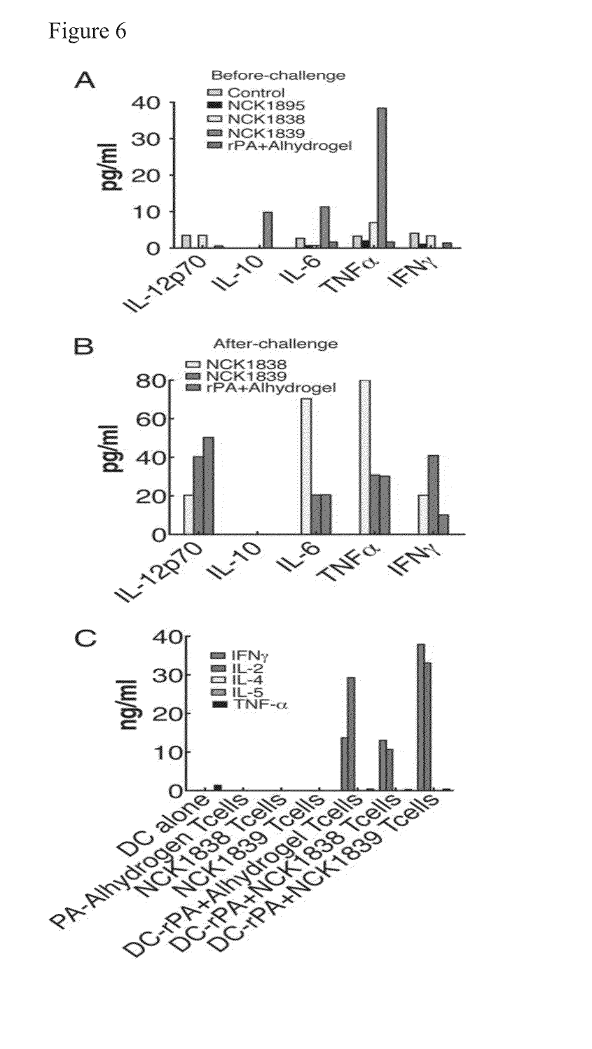

Cytokines and chemokines released into the peripheral blood of all mice were assayed as described above. Data show that L. acidophilus expressing PA-DCpep orally administrated into mice before challenge induced the up-regulation of IL-10, IL-6, TNFα, and MCP-1 (1.4 ng/mL) whereas L. acidophilus expressing PA-Ctrlpep induced increased levels of only TNFα (FIG. 6A). Furthermore, cytokines in the sera derived from mice that survived challenge by B. anthracis after vaccination with L. acidophilus expressing PA-DCpep showed trends before and after challenge, mainly in the production of IL-12, IL-6, TNFα, and IFNγ (FIG. 6B). IL-10 production was not sustained during the course of the infection in these mice (FIG. 6B). Although the production of IL-6, TNFα, and MCP-1 (43.7 pg/mL) was higher in mice that received L. acidophilus PA-Ctrlpep, IL-12p70, and IFNγ were conversely lower in these mice (FIG. 6B). As seen in FIG. 6B, IL-12p70, IL-6, TNFα, IFNγ, and MCP-1 (25 pg/mL) were induced at low levels in mice vaccinated s.c. with rPA plus alhydrogel; however, it rose significantly after Sterne challenge. Other cytokines such as IL-4 (≦0.1 pg/mL), IL-5 (≦0.2 pg/mL), and IL-2 (≦0.4 pg/mL) were expressed at very low levels in the sera of all vaccinated mice. Furthermore, data show that rPA fusion proteins expressed by L. acidophilus in vivo clearly elicited Th1 immune responses in mice that survived the Sterne challenge (FIG. 6C).

### Example 2

This example describes the induction of humoral and T cell immunity.

Expression of Rat/Neu Fused with a DC-Binding Peptide in Lactobacillus acidophilus.

Cloning of the Rat/neu tagged with a DC targeting or control fusion peptide into a high copy number vector for expression in L. acidophilus.

Previously, the PA tagged with a fusion DC targeting peptide (PA-DCpep) or control peptide (PA-Ctrl) was cloned into pTRK882, a low copy shuttle cloning vector that replicates in both E. coli and gram positive bacteria such as Lactobacillus species. The PA was successfully expressed in mice from a strong, constitutive ppg promoter from pTRK882. Oral administration of L. acidophilus NCFM cells containing the plasmid induced protective immunity in mice challenged with B. anthracis Sterne (Mohamadzadeh et al., PNAS, 2009). The expression system was further optimized in a high copy number vector (pTRK696), the L. acidophilus Vector pTRK696 that is based on a derivative of pNZ123. This cloning vector carrying a selectable chloramphenicol resistance gene and a rolling circle origin of replication functions in both E. coli and most gram positive bacteria. In addition, the 2.8 kb plasmid pTRK696 encodes a strong constitutive promoter, P6, that originates in L. acidophilus. Briefly, the genes for the Rat/neu-DCpep and Rat/neu-Ctrl fusions are PCR amplified from the original plasmids, using flanking primers similar to the ones used for cloning in pTRK882, except that they encoded XbaI and XhoI restriction sites at the 5′ and 3′ ends, respectively: Rat/neu forward XbaI and Rat/neu reverse XhoI. The PCR fragments are purified, digested with XbaI and XhoI, and ligated into similarly digested pTRK989. Plasmid clones are recovered in E. coli MC1061 and inserts are confirmed with restriction digests and sequencing. In addition to the cloning primers, Rat/neuI and Rat/neu II are used for sequencing. The designated plasmids are pTRK990 (Rat/neu-DCpep) and pTRK991 (Rat/neu-Ctrl). These plasmids are transformed into L. acidophilus NCFM by electroporation. Sequencing of PCR products from the transformants are confirmed with the presence of intact Rat/neu-DCpep and Rat/neu-Ctrl inserts downstream of the P6 promoter in L. acidophilus. Expression and localization of the Rat/neu-control peptide and Rat/neu-DCpep fusion is confirmed by Western blot analyses of bacterial lysates and culture media using a polyclonal antibody to Rat/neu protein.

Vaccination of the Mice with L. acidophilus Expressing Her2/neu-DCpep

Groups of transgenic FVB/N Her2/neu female mice (6-8 week of age, 20 mice/group) are inoculated intragastrically with live recombinant Lactobacillus acidophilus as follows: (1) Rat/neu-DC-pep fusion, (2) Rat/neu-control peptide fusion, (3) L. acidophilus harboring empty vector, and (4) a group of mice is used as a control group, which is treated with just PBS. The inoculums are 10⁸ colony-forming-units in 250 μl sterile PBS. The administration is repeated 3 times (day 0, 7, and 14) at weekly intervals. All groups of mice are boosted two weeks later. Prior to each boost and one month after the final boost, mice are bled to determine Ig-production against Rat/neu in the serum, and Rat/neu-specific T-cell proliferation and activation are analyzed. The Rat/neu-specific antibodies are analyzed by ELISA. For T-cell proliferation, DCs are loaded with recombinant Rat/neu protein or specific peptides synthesized as costume peptides by EZBiolab. Inc. (Westfield, Ind.) and co-cultured with CD4⁺ or CD8⁺ T cells derived from mesenteric lymph nodes and spleen, followed by an assay measuring T cell activation. The peptide sequences are as follows: PDSLRDLSVF; SEQ ID NO:5 (420-429), PYNYLSTEV; SEQ ID NO:6 (301-310), LFRNPHQALL; SEQ ID NO:7 (489-498), PGPTQCVNCS; SEQ ID NO:8 (528-537), PNQAQMRIL; SEQ ID NO:9 (712-720), GSGAFGTVYK; SEQ ID NO:10 (732-741), AFGTVYKGI; SEQ ID NO:11 (735-743), PYVSRLLGI; SEQ ID NO:12 (785-793), and LQRYSEDPTL; SEQ ID NO:13 (1,114-1,123). Once robust immune response are established, the experimental mouse groups are then given 5×10⁵ NT-2 tumor cells (S.C.) on the right flank on day 42. Subsequently, tumors are measured every two days for 100 days with calipers spanning the shortest and longest surface diameters. The NT-2 tumor cell line originated from a spontaneously occurring mammary tumor in FVB/N Her2/neu transgenic mice (Ercolini A. M, et al, J. Immunol, 2003). This cell line constitutively expresses low levels of rat Her2/neu antigen and is injected into transgenic mice to generate solid tumors. The NT-2 cell line is used as a feeder cell line as a source of antigen for the restimulation of splenic cells and mesenteric lymph node cells for CTL assay.

### CTL Assay.

To conduct CTLs, splenic and mesenteric lymph node cells are isolated from 6-8 weeks old female Her2/neu transgenic mice and FVB/N wild-type mice that are vaccinated with L. acidophilus expressing the immunogenic fusions and their controls. These cells are then co-cultured with irradiated NT-2 tumor cells (20,000 rads) at a ratio of 10:1 (splenic:tumor cells) with 20 U/ml IL-2. Subsequently, these cells are harvested and used in a standard CTL assay with 3T3 target cells loaded with specific target peptides (1 μg/ml). Afterwards, total lysates of chromium loaded target cells are stimulated by the addition of 2% Triton-X. Thereafter, the percent of specific lysis is calculated [%=100 (experimental lysis−spontaneous lysis)/(total lysis−spontaneous lysis)].

### Determination of the Immunotherapeutic Effects of Probiotic Strategy Against Rat/Neu In Vivo.

Groups of transgenic FVB/N Her2/neu female mice (6-8 week of age, 20 mice/group) are given 5×10⁵ NT-2 tumor cells (S.C.) on the right flank on day 0. Afterwards, when the tumor growth is visible (around day 14-21), these groups of mice are inoculated orally with Lactobacillus acidophilus expressing (1) Rat/neu-DC-pep, (2) Rat/neu-control peptide, (3) empty vector, and (4) a control group treated with just PBS. The inoculums are 10⁸ CFU in 250 μl sterile PBS. The administration is repeated 5 times at weekly intervals. Subsequently, tumors are measured every two days for 100 days as describe above and CTL assay and T cell immune responses are analyzed.

### Example 3

This example describes oral vaccination with L. gasseri expressing selected HA/NA/PA/HP/NP/NS/PB-immunogenic epitopes of H1N1 and H3N2 fused to DCpep.

Cloning of the HA/NA/NP selected peptides targeted with a DC-binding peptide or its control in L. gasseri. Expression vectors encoding the selected HA/NA/PA/HP/NP/NS/PB-immunogenic influenza A-epitopes-DCpep fusion and selected HA/NA/PA/HP/NP/NS/PB-immunogenic influenza A-epitopes-control peptide fusion are constructed and expressed in the common human commensal Lactobacillus gasseri. High-copy plasmids and strong promoters are used to maximize expression. The level of fusion protein expression, plasmid stability, and immunogenicity is analyzed. Integration vectors are employed to promote genetic stability of the expression cassettes, when appropriate. These steps deliver a highly-expressed HA/NA/PA/HP/NP/NS/PB-immunogenic influenza A-epitopes-DCpep fusion or its control fusion in vivo. L. gasseri provides a safe host for recombinant HA/NA/PA/HP/NP/NS/PB-immunogenic epitopes-DCpep production, which is manufactured, stored as a powder, and administered orally. Secretion by L. gasseri provides increased bio-availability of the HA/NA/PA/HP/NP/NS/PB-immunogenic epitopes-DCpep fusion since these bacteria present immunostimulatory components, such as cell wall (peptidoglycan), lipotechoic acid (LTA), and unmethylated DNA (CpG). These features promote the partial maturation of DCs and the production of IL-12 to induce potent influenza A antigen specific T cell immune responses against the viral challenge. Briefly, the encoding regions of the selected influenza epitopes-DCpep, or -Ctrl fusions are PCR-amplified from the original plasmids using flanking primers similar to those used for cloning in pTRK882, except that they encode XbaI and XhoI restriction sites at the 5′ and 3′ ends, respectively: Influenza A-selected immunogenic epitopes-DCpep forward XbaI and Influenza A-selected immunogenic epitopes-DCpep reverse XhoI. The PCR fragments are purified, digested with XbaI and XhoI, and ligated into similarly digested pTRK989. Plasmid clones are recovered in E. coli MC 1061 and inserts are confirmed with restriction digests and sequencing. In addition to the cloning primers, Influenza A-selected epitopes-DCpep, and their control fusions are used for sequencing. The designated plasmids are pTRK990 (Influenza A-selected epitopes-DCpep, Table 2) and pTRK991 (Influenza A-selected epitopes-Control pep). These plasmids are transformed into L. gasseri by electroporation. Sequencing of PCR products from the transformants is confirmed with the presence of intact Influenza A-selected epitopes-DCpep and Influenza A-selected epitopes-Control peptide inserts downstream of the P6 promoter in L. gasseri. Expression and localization of both fusions is confirmed by Western blot analyses of bacterial lysates and culture media using a polyclonal antibody to DC-peptide or its corresponding control protein.

TABLE 2
Highly immunogenic influenza A
CD4⁺ and CD8⁺ T cell peptides.

| | | |
|---|---|---|
| HA 211-225 | YVQASGRVTVSTRRS; | SEQ ID NO: 14 |
| HA 261-275 | INSNGNLIAPRGYFK; | SEQ ID NO: 15 |
| HA 276-290 | MRTGKSSIMRSDAPI; | SEQ ID NO: 16 |
| HA 321-335 | CPKYVKQNTLKLATG; | SEQ ID NO: 17 |
| HA 326-340 | KQNTLKLATGMRNVP; | SEQ ID NO: 18 |
| HA 441-455 | AELLVALENQHTIDL; | SEQ ID NO: 19 |
| HA 446-460 | ALENQHTIDLTDSEM; | SEQ ID NO: 20 |
| HA475-482 | KEIGNGCFEF/Db; | SEQ ID NO: 21 |
| NA181-189 | SGPDNGAVAV/Db; | SEQ ID NO: 22 |
| NA335-343 | YRYGNGVWI/Db; | SEQ ID NO: 23 |
| NA425-432 | SSISFCGV/Kb; | SEQ ID NO: 24 |
| NP 136-150 | MMIWHSNLNDATYQR; | SEQ ID NO: 25 |
| NP 151-165 | TRALVRTGMDPRMCS; | SEQ ID NO: 26 |
| NP 161-175 | PRMCSLMQGSTLPRR; | SEQ ID NO: 27 |
| NP 196-210 | MIKRGINDRNFWRGE; | SEQ ID NO: 28 |
| NP 201-215 | INDRNFWRGENGRKT; | SEQ ID NO: 29 |
| NP 206-220 | FWRGENGRKTRIAYE; | SEQ ID NO: 30 |
| NP 211-225 | NGRKTRIAYERMCNI; | SEQ ID NO: 31 |
| NP 216-230 | RIAYERMCNILKGKF; | SEQ ID NO: 32 |
| NP 311-325 | QVYSLIRPNENPAHK; | SEQ ID NO: 33 |
| NP 316-330 | IRPNENPAHKSQLVW; | SEQ ID NO: 34 |
| NP366-374 | ASNENMETM; | SEQ ID NO: 35 |
| HP43-50 | GGLPFSLL; | SEQ ID NO: 36 |
| NS2114-121 | RTFSFQLI; | SEQ ID NO: 37 |
| NS1133-140 | FSVIFDRL; | SEQ ID NO: 38 |
| PA 276-290 | CSQRSKFLLMDALKL; | SEQ ID NO: 39 |
| PA 316-330 | GWKEPNVVKPHEKGI; | SEQ ID NO: 40 |
| PA224-233 | SSLENFRAYV; | SEQ ID NO: 41 |
| PA238-245 | NGYIEGKL; | SEQ ID NO: 42 |
| PA300-307 | GIPLYDAI; | SEQ ID NO: 43 |
| PB2 91-105 | VSPLAVTWWNRNGPM; | SEQ ID NO: 44 |
| PB2 106-120 | TNTVHYPKIYKTYFE; | SEQ ID NO: 45 |
| PB2 196-210 | CKISPLMVAYMLERE; | SEQ ID NO: 46 |
| PB1214-221 | RSYLIRAL; | SEQ ID NO: 47 |
| PB2358-365 | GYEEFTMV; | SEQ ID NO: 48 |
| PB2689-696 | VLRGFLIL; | SEQ ID NO: 49 |

Vaccination of Mice with L. Gasseri Expressing Influenza a Selected Epitopes-DCpep Fusion.

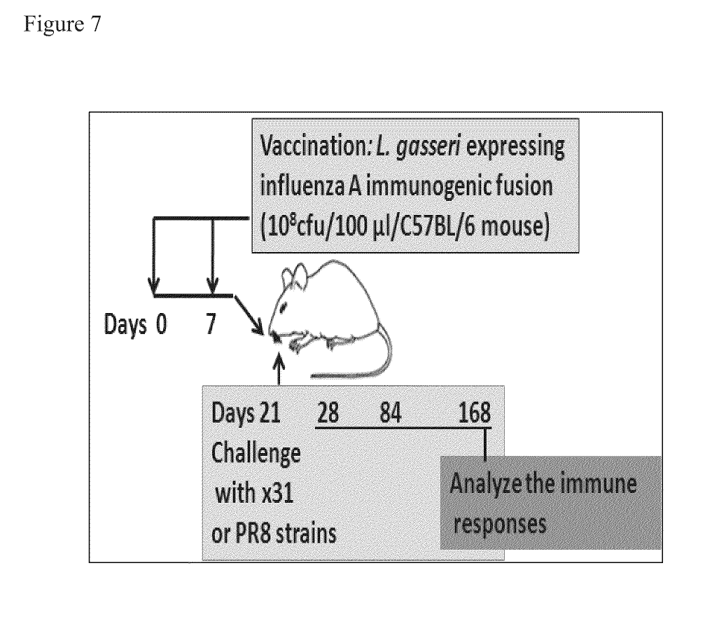

Groups of C57BL/6 (B6) mice (age 5-8 wk, female, 50 mice/group) are inoculated (10⁸ colony-forming-units in 100 μl) intragastrically with live recombinant L. gasseri expressing Influenza A-selected epitopes-DCpep, Influenza A selected epitopes-Control pep, L. gasseri harboring empty vector, and a group of PBS-treated control mice are used as control groups. The administration of L. gasseri expressing the vaccine fusion is repeated twice (day 0, and 7) at weekly intervals. Prior to each boost, mice are bled to determine antibody-production against Influenza A-selected epitopes-DCpep fusion in the serum of the mice. The influenza virus strains HK-×31 (×31; H3N2 or A/PR8/34 (PR8, H1N1) are grown, stored and iterated as previously described (Crowe S. R., 2005, Vaccine). At day 21, mice are anesthetized by i.p. injection of 2,2,2-tribromoethanol and infected intranasally (i.n) with 300 or 600 50% egg infectious dose (EID50) of influenza A strains ×31 or PR8. Animal weight and survival are monitored through day 168. Humoral and T cell mediated immune responses are analyzed on the following days: 28, 84 and 168. Humoral antibody production and the numbers, quality, and anatomical distribution of influenza A specific CD4⁺ and CD8⁺ T cells is assayed using a variety of highly sensitive techniques, including Enzyme-linked immunospot assay (ELISPOT), ELISA, CTL assay, as well as FACS analysis using both intracellular and tetramer staining as described previously (Crowe S. R., 2005, Vaccine). Additionally, viral titer and animal survival is also determined (FIG. 7). FIG. 7 shows depiction of vaccination, infection and analysis of immune responses of mice to be infected with ×31 or PR8 Influenza A strains.

### ELISpot and Intracellular Staining.

The numbers of IFNγ-secreting cells derived from spleens and lung airways of infected mice is determined after stimulation with Influenza peptides using a standard ELISpot assay (Crowe S. R., 2005, Vaccine). Additionally, intracellular cytokine staining and FACS analysis is performed. Briefly, lymphocytes are collected from the spleens or lung airways (broncoalveloar lavage) as previously described (Crowe S. R., 2005, Vaccine). Collected cells (10⁶/condition) are stimulated with influenza A peptides (10 μg/250 μl) in the presence of IL-2 and Brefeldin at 37° C. for 5 hrs. Subsequently, cells are stained with anti-CD4 FITC, anti-CD8 PerCep, and anti-CD44 PE. The cells are fixed, permeabilized, and stained with anti-IFNγ PE and analyzed by FACS.

### ⁵¹Cr Release Assay (CTL).

To generate X31 or PR8-infected target cells, mouse EL-4 lymphoma cells (2×10⁶) are resuspended in serum-free RPMI medium (400 μl). The cells are then incubated with ×38 or PR8 viral particles for 1 hr at 37° C. Virally infected cells are transferred to 6-well plates containing 6 ml of cRPMI medium/well and incubated overnight. To generate peptide-pulsed target cells, EL-4 cells (10⁶) are incubated with individual influenza peptides (20 μg/ml) in 500 μl of complete RPMI medium for 1 h at 37° C. Both ×38 and PR8-infected and peptide-pulsed EL-4 target cells are washed and then labeled with ⁵¹Cr (150 μl) for 90 min at 37° C. Unpulsed ⁵¹Cr-labeled EL-4 cells are used as control target cells. After washing three times, target cells (10⁴) are incubated with titrated concentrations of effector CD8⁺ T cells in a final volume of 200 μl. Supernatants (100 μl) are removed after 5 hrs incubation for γ-radiation counting.

Intranasal Vaccination with Selected HA/NA/PA/HP/NP/NS/PB-Immunogenic Epitopes of H1N1 and N3N2 Fused to DCpep Delivered with an Adjuvant

Killed L. gasseri plus immunogenic fusion protein containing Influenza A selected epitopes-DCpep, or -control pep are applied intranasally. Briefly, groups of C57BL/6 (B6) mice (age 5-8 wk, female, 50 mice/groups) are vaccinated twice, on days 0, and 7, at a dose of 20 μg of immunogenic fusion proteins combined with killed L. gasseri that is diluted to a final concentration of 2×10⁹ CFU/ml and heat inactivated for 10 min at 70° C. and subsequently applied intranasally (50 μl/mouse). Blood and fecal extracts are collected every week to measure serum IgG and secretory IgA. Fecal pellets are dissolved in PBS and centrifuged for further analysis. Six weeks after the second inoculation, anesthetized mice are infected i.n with either 300 or 600 50% egg infectious dose (EID50) of influenza A strains ×31 or PR8. The percentage of animals surviving is observed over a period of 168 days. ELISPOT, ELISA, CTL, as well as FACS analysis using intracellular and tetramer staining are conducted as described above. Additionally, viral titer and animal survival is also determined.

### Example 4

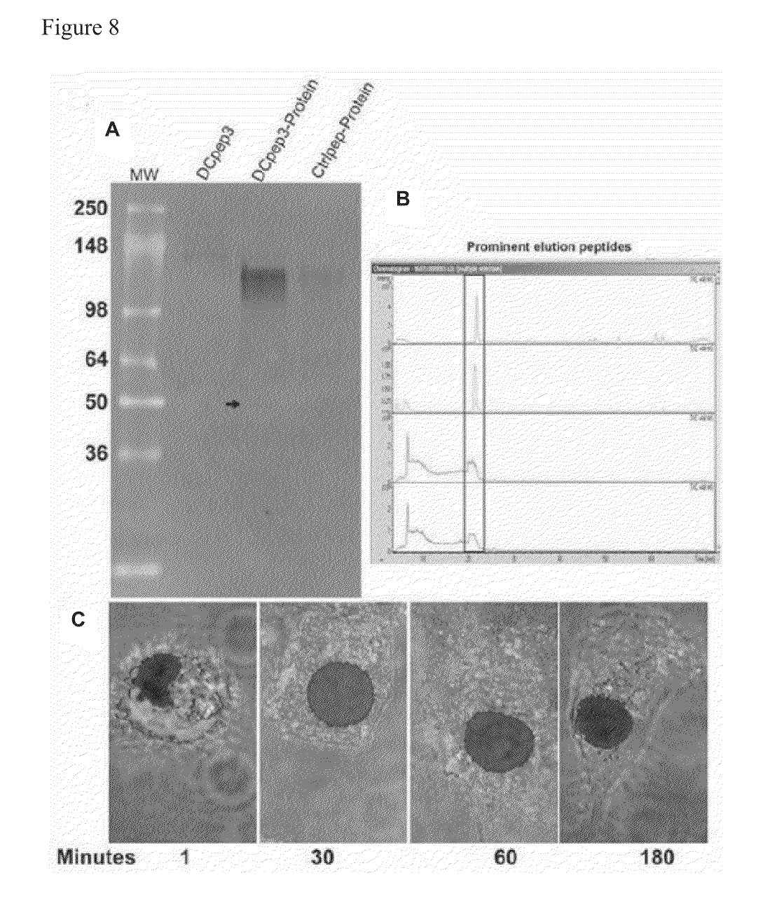

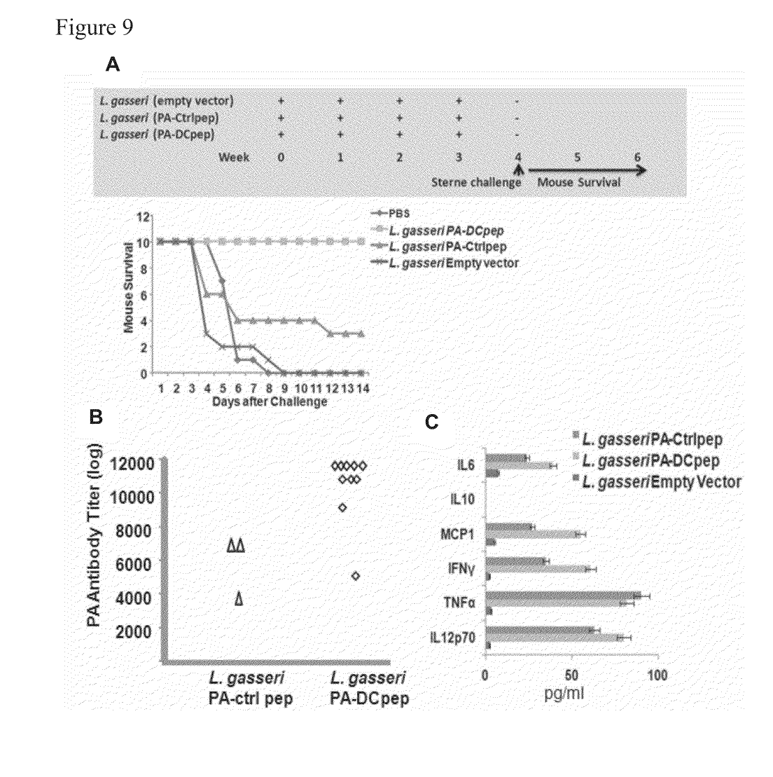

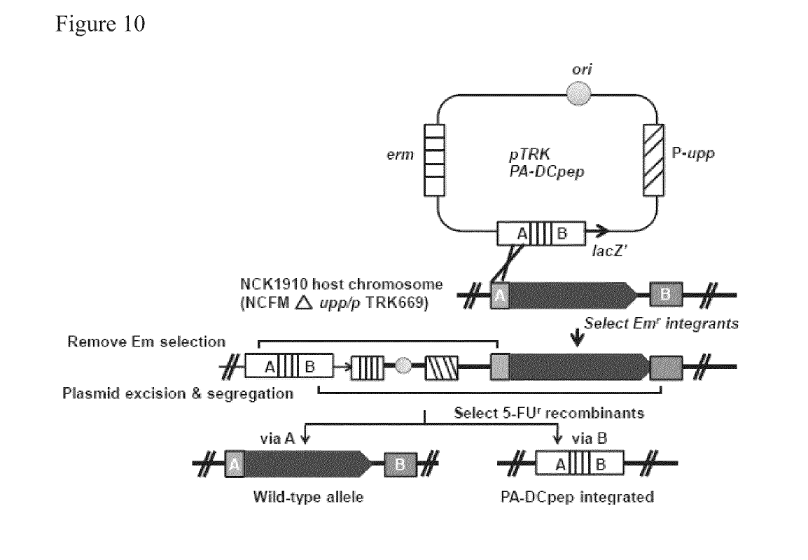

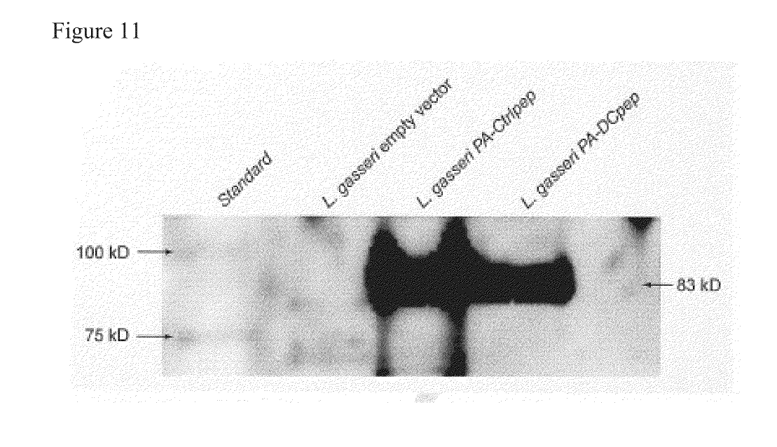

This example describes L. gasseri expressing targeted PA-DCpep or Hc-DCpep fusion vaccines. FIG. 10 shows an exemplary markerless gene replacement strategy for insertion of PA-DCpep or Hc-DCpep into a targented region of a bacterial genome. Several 12-mer peptides derived from a phage display peptide library have been identified. Receptor saturation studies shows that these peptides bind to different surface molecules and the interaction with their ligands does not impair the immunobiology of DCs. One of these peptides (DCPeptide#3) was selected because it bound most efficiently to human, nun-human primate (NHP), and mouse DCs. Characterization of the ligand to which DC-peptide 3 binds (DCpep) shows a distinctive band (50 kDa) that was analyzed by liquid chromatography mass spectrometry (LC-MS). Sequence analysis revealed a novel candidate binding receptor protein that is actively involved in the endocytotic pathway of DCs (FIG. 8). To show its efficacy for vaccine, the encoding sequence of this DCpep was genetically fused with B. anthracis PA and cloned into a low copy cloning vector and expressed in L. acidophilus. Expression of PA-DCpep by L. acidophilus in the gut clearly induced protection against anthrax Sterne challenge. To improve the efficacy of PA-DCpep, a stable vector with a strong promoter was adapted and expressed in L. gasseri. L. gasseri expressing PA-DCpep was 100% efficacious in protection of the mice that were infected with Sterne (FIG. 9). These data thus demonstrate that by using this high copy expression system the oral vaccine strategy does not require as many L. gasseri cells expressing the PA-DCpep fusion as was required previously using a low copy number expression vector in L. acidophilus (10⁸ cfu/100 μl compared to 10⁹ cfu in 250 μl). Moreover, the vaccination period was shorter (4 vaccinations compared to 4 vaccinations plus 2 boosters in previous work). Employing L. gasseri as a delivery vector was not only efficacious but also served as an excellent adjuvant to induce solely IL-12 in DCs, as previously demonstrated. The cellular binding domain of BoNT/A-Hc has been identified as a vaccine candidate containing a β-trefoil structure. That is a structure common to all BoNT-serotypes. This motif is repeated in the progenitor toxin complex, indicating that vaccination with Hc is sufficient to elicit neutralizing Abs to protect against BoNT intoxications.

L. gasseri Expression of Targeted PA Fusion.

Briefly, preimmune blood samples from mice are collected and stored. Mice (C57BL/6, 6-8 weeks old, 20/each group) are vaccinated orally with live L. gasseri (108 CFU/100 μl of PBS) for four consecutive weeks as follows: 1) L. gasseri-empty vector, 2) L. gasseri-PA-Ctrlpep, 3) L. gasseri-PA-DCpep. Additionally, mice (12/group) are injected with 25 μg of rPA plus 0.3% aluminum hydroxide gel as an adjuvant. Mice are bled for PA antibody titer around day 28. These mice are challenged on day 35. After the third vaccination, mice are bled to determine serum anti-PA antibody levels. The neutralizing PA specific antibodies are analyzed by ELISA. On day 35, mice are split in two subgroups of 10 mice each for anthrax inhalation. Each subgroup of mice is challenged with 9602 strain (10 to 15 LD50) or 17JB (10 to 15 LD50) of B. anthracis. Briefly, spores are diluted to a final concentration of 2×10⁹ CFU/ml for 10 minutes at 70° C. Mice are anesthetized, and 50 μl of these bacteria is administrated intranasally at 10⁸ CFU/mouse. Mouse survival is monitored over time. These experiments are performed using C57BL/6 mice that are susceptible to anthrax 9602 (virulence equivalent to Ames strain) and 17JB (virulence equivalent to Vollum1B) infection.

L. gasseri Expressing BoNT/A-Hc-DCpep.

To clone BoNT/A-Hc-DCpep, the stable theta replicating, erythromycin resistant shuttle vector, pTRKH2, is evaluated as a suitable and high expression cloning vector for the Hc-DC antigen in L. gasseri. In a two step process, PCR cloning is used to amplify the Hc-DCpep and Hc-Ctrlpep synthesized genes and insert them, along with the constitutive Lactobacillus promoter, P6 into pTRKH2 digested with BamHI/XhoI. Plasmid clones are recovered in E. coli DH5α, and inserts are confirmed by restriction digest patterns and DNA sequencing. In addition to the M13 primers flanking the multiple cloning site of pTRKH2. The plasmids are then transformed into L. gasseri by electroporation. The L. gasseri strains expressing Hc-DCpep or Hc-Ctrlpep are designated and used for animal vaccination. Briefly, mice (BALB/c and C57BL/6, 6-8 weeks old, 20/each group) are inoculated intragastrically with live L. gasseri (10⁸ CFU/100 μl of PBS) for 4 consecutive weeks as follows: 1) L. gasseri empty vector, 2) L. gasseri-Hc-Ctrlpep, 3) L. gasseri-Hc-DCpep, and 4) PBS. For positive protection, mice (n=10/group) are nasally vaccinated with 50 μg Hc/A or Hcβtre plus CT as adjuvant (2 μg) on days 0, 7, 14, and 28. In addition, Hc on alum is given via the i.m. route on days −7, 0, 14, and 28. Sera from each group of mice are analyzed for anti-rHc antibodies by ELISA. Mice from all groups are challenged with pure BoNT/A (Metabiologics) diluted in PBS containing 0.2% (w/v) gelatin 26 2 weeks after the last vaccination. The mice are observed for 2 weeks after challenge, and animal survival is determined for each group of mice.

### Chromosomal Integration of PA-DCpep or Hc-DCpep in L. Gasseri.

A site directed integration and gene deletion system has been developed for L. gasseri and L. acidophilus. That system has been successfully used to generate numerous gene knockouts and deletions for functional genomics analysis in the L. acidophilus. Recently, this system was significantly improved for selecting gene deletions by providing a positive selection marker to detect excision events of the integration plasmid in a second recombination event. The expression host background is L. gasseri with a deletion in the uppencoded uracil phosphoribosyltransferase. This makes the upp-deletion strain resistant to 5-fluorouracil (5-FU), and other phenotypic changes were identified. The integration vector used in this study encodes a functional upp gene and the anthrax PA-DCpep or BoNT/A Hc-DCpep genetic cassette, flanked by two regions in the genome that are being targeted for the first and second integration events. Initial transformants and integrants in L. gasseri are erythromycin (Em)-resistant and 5-FU sensitive. Propagation of those clones in the absence of Em results in excision and loss of the targeting vector, and those derivative clones are then Em-sensitive and 5FU resistant. Those clones undergoing the second excision event are positively selected on media containing 5-FU and screened for the desired gene replacement with the PA-DCpep, Hc-DCpep, or its control genetic cassette. Clones with the proper genetic characteristics are screened by Western blot for expression of PADCpep, Hc-DCpep or their controls using specific antibodies for PA or Hc.

Efficacy of Chromosomal Integration of PA-DCpep Fusion Protein In Vivo.

To validate the efficacy and the expression of the immunogenic fusion by chromosomal integration, groups of A/J mice are used. Briefly, these groups of mice are orally inoculated with L. gasseri empty vector (10⁸ CFU in 100 μl sterile PBS), L. gasseri expressing PA-DCpep, and L. gasseri expressing PA-Ctrlpep for four consecutive weeks. Additionally, mice (10/group) are injected with 25 μg of rPA plus 0.3% aluminum hydroxide gel as an adjuvant. Seven days after the last vaccination, mice are challenged with B. anthracis Sterne (5×10⁴CFU/mouse/500 μl/i.p). Subsequently, mouse survival is monitored over time, as described above. Total and neutralizing anti-PA antibodies are analyzed, as described above. After confirming the expression and the efficacy of PA-DCpep, when expressed by chromosomal insertion, the following experiments are performed.

Sterne (pXO1+/pXO2−).

Livestock vaccines used for vaccination against B. anthracis are derivatives of the live spore vaccine formulated by Sterne in 1937. While toxin and capsule producing wild-type strains harbor the two virulence plasmids pXO1 (toxin plasmid codes for the three toxin proteins: PA, LF and EF) and pXO2, which codes for the polypeptide capsule, the Sterne strain contains only pXO1, rendering it oxygenic yet avirulent when administered to most animals. However, as reported by Welkos et al., several species of inbred mice, including A/J mice, remained susceptible to the oxygenic Sterne strain used in this study. For the basis of the experiments described below, the Sterne strain, which lacks the plasmid pXO2 and without its capsule phagocytosis/opsonization is severely perturbed in vitro is used.

Inhaled anthrax. Groups of C57BL/6 mice (n=20/group) are used as follows: 1) L. gasseri empty vector, 2) L. gasseri PA-DCpep, and 3) L. gasseri PA-Ctrlpep. Briefly, these groups of mice are orally inoculated with L. gasseri, as outlined above, for four consecutive weeks. Prior to challenge, mice are bled to confirm presence of anti-PA antibody titers. Additionally, mice (10/group) are injected with 25 μg of rPA plus 0.3% aluminum hydroxide gel as an adjuvant. This group of mice serves as a positive control group. Seven days after the last vaccination, all groups of mice are challenged with 9602 B. anthracis, as described above. Subsequently, mouse survival is monitored over time, as described above. Total and neutralizing anti-PA antibodies are analyzed. It is contemplated that the chromosomal insertion of PA-DCpep in L. gasseri provides protection against anthrax challenge.

### Chromosomal Integration (CI) of Hc-DCpep Fusion Protein In Vivo.

To evaluate the protective efficacy of the L. gasseri expressing Hc-DCpep vaccine, BALB/c and C57BL/6 mice (10/group) are orally vaccinated, as described above, once weekly for 4 wks, and these challenge studies are performed twice. Challenge studies are performed using the identical vaccination protocol, as described above, to determine if the vaccines induce protective immunity against BoNT/A. These are done before additional immunogenicity studies are performed. For positive protection, mice are nasally vaccinated with 50 μg Hc/A or Hcβtre plus CT (2 μg) on days 0, 7, 14, and 28. In addition, Hc on alum is given via the i.m. route on days −7, 0, 14, and 28. Negative control mice include mice orally vaccinated with L. gasseri empty vector, L. gasseri Hc-Ctrlpep, and naïve mice. Prior to challenge, plasma and fecal samples are collected on days 21 and 28 to confirm by ELISA the presence of Hc-specific IgG and IgA Abs. Hc-specific IgG and IgA endpoint titers of all groups are statistically compared by analysis of variance (ANOVA) followed by comparison of multiple means procedures when ANOVA analysis identifies significant differences to determine if differences between groups are statistically significant. Vaccination protocol is modified depending upon Ab levels achieved. When Ab titers for Hc/A are in excess of 216, mice are challenged. On day 35, mice are challenged by the i.p. route with 2,000 or 20,000 LD50 BoNT/A delivered in PBS containing 2 mg/ml gelatin. Mice are monitored daily for up to 7 days; body weight and activity (signs of paralysis) is monitored daily. Significance in protection is discerned at the 95% confidence interval.

B Cell Immunogenicity of the L. gasseri Expressing Hc-DCpep Vaccines for BoNT/A.

Once the ability of the Hc. L. gasseri expressing Hc-DCpep vaccine to induce protective immunity has been verified, studies are done to determine the source of mucosal S-IgA and plasma IgG Abs to Hc. The vaccines are administered to groups consisting of five mice each, and the experiments are repeated at least twice. From these studies, it is discerned whether oral vaccination with L. gasseri expressing Hc-DCpep vaccine enhances mucosal immunity in contrast to conventional peripheral (i.m. or s.c.) Hc vaccination using alum and mice vaccinated with L. gasseri empty vector or L. gasseri Hc-Ctrlpep. Plasma and mucosal secretions from vaccinated mice are obtained at day 21 in order to detect Hc-specific Ab responses. Nasal washes are done at the termination of the study and taken from mice used for B cell ELISPOT analysis of Ab-forming cell (AFC) responses. For some groups, titers of IgG and IgA Abs are also monitored for six months following the last vaccination to determine the longevity of these Ab responses. Peak plasma Ab titers are evaluated for the IgG subclasses.

Validation of T Cell Immunogenicity of L-gasseri Expressing PA-DCpep and Hc-DCpep Vaccines for Anthrax and BoNT/A.

Subsequent studies determine which Th cell subsets(s) are responsible for protection. Initially, mice are vaccinated according the regimen described above. Between days 35 and 42, CD4⁺ T cells (isolated by flow cytometry using a Beckton-Dickinson FACSAria) from spleen, mesenteric LNs, Peyer's patches, and regional lymph nodes are co-cultured with irradiated bone marrow derived DCs, either without or with 10 μg recombinant Hc 44, 10 μg rPA58, or with an irrelevant antigen, 1.0 mg OVA 59,60, for 3-5 days. During the last 16 hours of culture, some of the supernatants are collected for cytokine analysis and subsequently cells are pulsed with ³H-thymidine to examine level of incorporation in response to each vaccine. From these studies, it is expected that one observe enhanced responsiveness by the CD4⁺ T cells from vaccinated mice when compared to CD4⁺ T cells from control (empty L. gasseri vector- or L. gasseri PA- or Hc-Ctrlpep-dosed) mice. Subsequent CD4⁺ T cell analysis determines whether the CD4⁺ T cells exhibit a Th1, Th2, or Th17 cell bias. To distinguish among these Th cell subsets, cytokine-specific ELISA/ELISPOT assays are used: the Th1 cell cytokines, IL-2 and IFN-γ; Th17 cell cytokines IL-6, IL-17, and IL-21; and the Th2 cell cytokines IL-4, IL-5, IL-10, IL-13, and TGF-β. FACS analysis are performed to determine the CD4⁺ T cell subsets present and the source of the cytokine-producing cells. Thus, from these cytokine analyses, it is determined whether there is a preferential Th cell bias or a mixed Th cell response. In either case, it is determined which Th cell(s) account for the efficacy of the designed vaccination regimen.

Efficacy of the Multivalent Vaccine of PA+Hc-DCpep Fusion Against Inhalational Anthrax and BoNT/A when Expressed from a Chromosomal Location in L. gasseri.

Expression of Multivalent Vaccine by L. gasseri and Vaccine.

The encoding sequences of both immunogenic subunits fused to DCpep or their controls is expressed in L. gasseri, as described above. This multivalent vaccine platform is validated using Sterne infection as described above. Mice are vaccinated with L. gasseri expressing 1) PA-Hc-DCpep, 2) PA-Hc-Ctrlpep, or 3) empty vector for four consecutive weeks. Additionally, positive control groups of mice (n=10 mice/group) are used for PA and BoNT/A vaccination as described above. Before exposing the animals to pathogens, mice are bled to determine neutralizing anti-PA and anti-Hc antibodies in their sera. Mice are first challenged using pure BoNT serotype A diluted in PBS containing 0.2% (w/v) gelatin 26 two weeks after the last vaccination. The mice are observed for two weeks after challenge, and animal survival is determined for each group of mice, as described above. Two months later, mice are then challenged with 9602 strain of B. anthracis, as described above. Mouse survival is monitored for two weeks.

### Example 5

### Material & Methods

Cloning of the PA-DCpep fusion protein. Previously, the synthesized gene for B. anthracis protective antigen, with its signal sequence for secretion and tagged with a PA-DCpep (FYPSYHSTPQRP; SEQ ID NO:1) or control peptide (PA-Ctrlpep: EPIHPETTFTNN; SEQ ID NO:2) was cloned into a low copy cloning vector and expressed in L. acidophilus NCFM (Mohamadzadeh et al., PNAS 106:4331 (2009)). For this study, the stable θ replicating, erythromycin resistant shuttle vector, pTRKH2, was evaluated as a suitable and high expression cloning vector for the PA-DCpep antigen in L. gasseri (O'Sullivan et al., Gene 137:227 (1993)). In a two step process, PCR cloning was used to amplify the PA-DCpep and PA-Ctrlpep synthesized genes and insert them, along with the constitutive Lactobacillus promoter, P6 (Djordjevic et al., Can. J. Microbiol. 43:61 (1997)) into pTRKH2 digested with BamHIIXhoI. The P6 promoter, isolated from L. acidophilus ATCC 4356, is a relatively strong promoter functional in E. coli, lactococci and lactobacilli (Djordjevic et al., supra). Erythromycin (EM)-resistant plasmid clones were recovered in E. coli DH5a, and inserts were confirmed by restriction digest patterns and DNA sequencing. In addition to the M13 primers flanking the multiple cloning site of pTRKH2, primers pagl (ATTAGGTGCAAGTATTTGAC; SEQ ID NO: 62) and pagll (AATACCGCTGATACAGCAAG; SEQ ID NO: 63) were used for sequencing. The plasmids were designated pTRK994 (PA-DCpep) and pTRK995 (PA-Ctrlpep). The plasmids were transformed into L. gasseri ATCC33323 by electroporation (Goh et al., Environ. Microbiol. 75:3093 (2009)). The L. gasseri strains expressing PA-DCpep or PA-Ctrlpep were then designated as NCK2065 and NCK2066. Western blots. To examine for recombinant (r) PA expression by L. gasseri, cultures expressing the PA-Ctrlpep and PA-DCpep, and L. gasseri harboring the empty vector were propagated to mid-log phase in deMan, Rogosa, and Sharpe broth (MRS; Difco, Detroit, Mich., USA) supplemented with 5 μg/ml EM. Proteins from culture supernatants were precipitated using trichloroacetic acid (TCA) and recovered by centrifugation. Total proteins from culture supernatants were loaded onto a 4-12% SDS-PAGE gel. After electrophoresis, the proteins were transferred to a nitrocellulose membrane and probed with anti-PA antibody conjugated with horseradish peroxidase (HRP). Transfer blot membranes were then washed, treated with Supersignal® West Femto substrate (Thermo, Rockford, Ill., USA), and visualized by a luminescent image analyzer LAS-3000 (Hanover Park, Ill., USA). The Precesion Plus Protein™ standard (Bio RAD, Hercules, Calif., USA) was used as the molecular weight marker.

In vivo vaccination. A/J mice used were 6- to 8-week-old A/J and were purchased from Jackson Laboratories (Bar Harbor, Me., USA). Experiments were performed in an accredited facility according to NIH guidelines in the Guide for Care and Use of Laboratory Animals. Animal protocols were approved by the local ethics committee. L. gasseri expressing PA-DCpep, PA-Ctrl pep and an empty vector control were grown at 37° C. in MRS broth supplemented with EM (5 μg/ml) for 72 h. Cells were centrifuged, washed twice in PBS, and resuspended in PBS at 10⁹ colony forming unit (CFU)/ml. Subsequently, groups of mice were orally vaccinated with 100 μl (10⁸ cfu) L. gasseri expressing PA-DCpep, PA-Ctrlpep, or cells harboring the empty vector. Oral vaccination was administered four times on a weekly basis. Additionally, mice (n=3) were used as a historical positive control, which were vaccinated with rPA adsorbed to alhydrogel by a single subcutaneous injection. One week later, the groups of mice were challenged intraperitoneally with B. anthracis Sterne pXO1⁺/pX0²⁻ (5×10⁴ CFU/mouse) (Welkos et al., Infect. Immunol. 51:795 (1986)). Survival was monitored until day 14. Additionally, blood was taken from each mouse before and after challenge to determine the levels of PA-neutralizing antibodies, and cytokines released into the peripheral blood, as described previously (Mohamadzadeh et al., PNAS 106:4331 (2009)). Statistical significance of survival was determined using GraphPad Prism v4.03.

Anti-PA antibody analysis. To determine the levels of neutralizing antiPA antibodies elicited by L. gasseri expressing PA-DCpep versus its controls, a toxin neutralization assay was utilized (Albrecht et al., Infect. Immunol. 75:5425 (2007)). Briefly, serially diluted sera derived from surviving mice from each group were incubated at 37° C. with B. anthracis lethal toxin (PA 100 ng/ml and LF 20 ng/ml). After 1 h, the mixture was added to J774A.1 macrophages (10⁵/well) in a 96-well plate. After 4 h incubation at 37° C., 25 μl of 3-(4,5-Dimethylthiazol-2-yl)-2,5-diphenyltetrazolium bromide, a tetrazole) (MTT; 1 mg/ml) dye was added and the cells were further incubated for 2 h. The reaction was stopped by adding an equal volume of lysis buffer (50% DMF and 20% SDS, pH 7.4). Plates were incubated overnight at 4° C. and the absorbance was read at 570 nm in a multiwell plate reader.

PA-specific T-cell stimulation. Bone marrow-derived DCs were prepared as described previously (Pulendran et al., Eur. J. Immunol. 34:66 (2004)). The rPA-treated, and untreated, DCs (10⁴/well) were seeded at graded doses in round-bottomed micro titer plates and subsequently cultured for 12 h at 37° C. T cells (10⁵/well) from mice that survived the B. anthracis Sterne challenge were isolated from mesenteric lymph nodes using a negative bead method (Mohamadzadeh et al., PNAS 106:4331 (2009)). These cells were co-cultured with PA-treated or untreated DCs for 5 days. Afterwards, cell supernatants were harvested and cytokine release analyzed using CBA mouse TH1/TH2 kits on the FACS Cantoll flow cytometer (BD, San Diego, Calif., USA). Co-cultures were then pulsed for the last 16 h with 0.5 μCi 3H thymidine/well (New England Nuclear, Del., USA) to assay T cell proliferation (Pulendran et al., supra).

### Results

Expression of targeted anthrax PA by L. gasseri. To improve the efficacy of PA-DCpep, a stable vector with a strong promoter was adapted and expressed in L. gasseri. Data show that after electroporation of pTRK994 (PA-DCpep) and pTRK995 (PA-Ctrlpep) into L. gasseri, high protein expression of PA-DCpep and the control peptide (6-10 μg/ml as measured by Bicinchoninic Acid protein assay; Thermo scientific, IL, USA) were detected, while PA was not detected in the supernatants of L. gasseri bearing the base vector without the PA-DCpep, or PA-Ctrlpep cassettes by Western blot (FIG. 11).

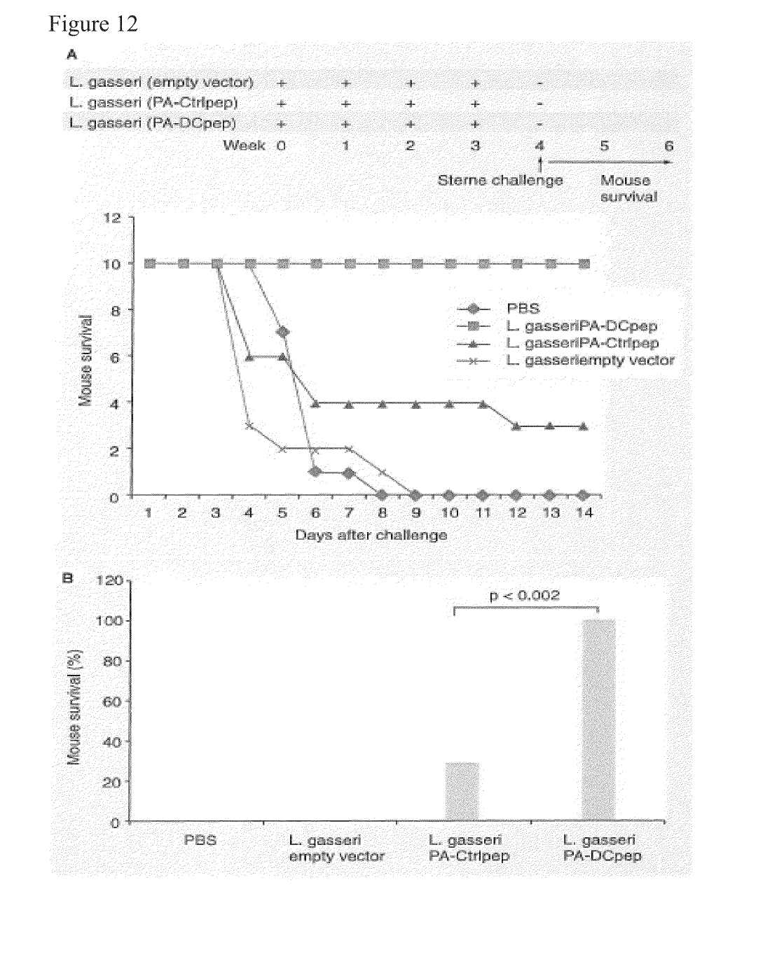

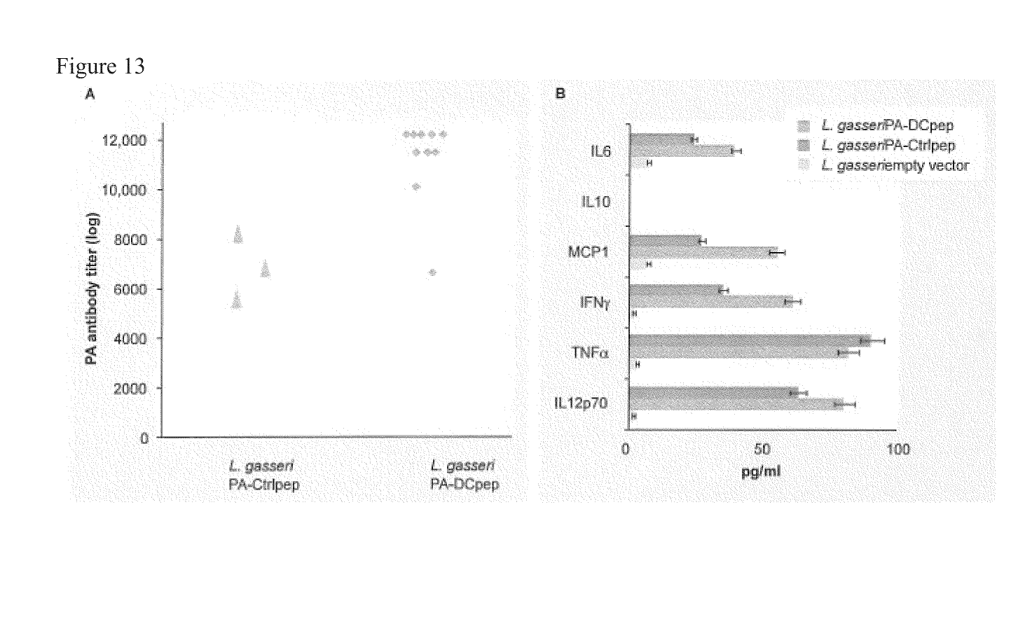

Induction of robust immune responses against anthrax infection. To test the efficacy of the high copy expression vector for PA-DCpep fusion in L. gasseri, groups of mice (n=10/group) were vaccinated with L. gasseri (10⁸/cfu), bearing the empty vector, L. gasseri expressing PA-Ctrlpep, or L. gasseri PA-DCpep for four consecutive weeks (FIG. 12A). On week four, all groups of mice were challenged with Sterne (5×10⁴/mouse/intraperitoneal) and mouse survival was monitored. L. gasseri expressing PA-DCpep fusion was 100% efficacious in protection of the mice compared with 30% survival (p<0.002) when vaccinated with L. gasseri expressing PA-Ctrl pep (FIGS. 12A & B). Additionally, vaccinated mice with rPA plus alhydrogel were fully protected from Sterne lethal challenge. Administration of PA-DCpep fusion by L. gasseri elicited robust toxin neutralizing antibody titers that were reported as the reciprocal of the dilution in the assay (FIG. 13A). Additionally, this oral vaccine platform also induced higher inflammatory cytokines (IL-6, IL-12, or IFNγ) and chemokine (MCP1) in the periphery, including blood (FIG. 13B).

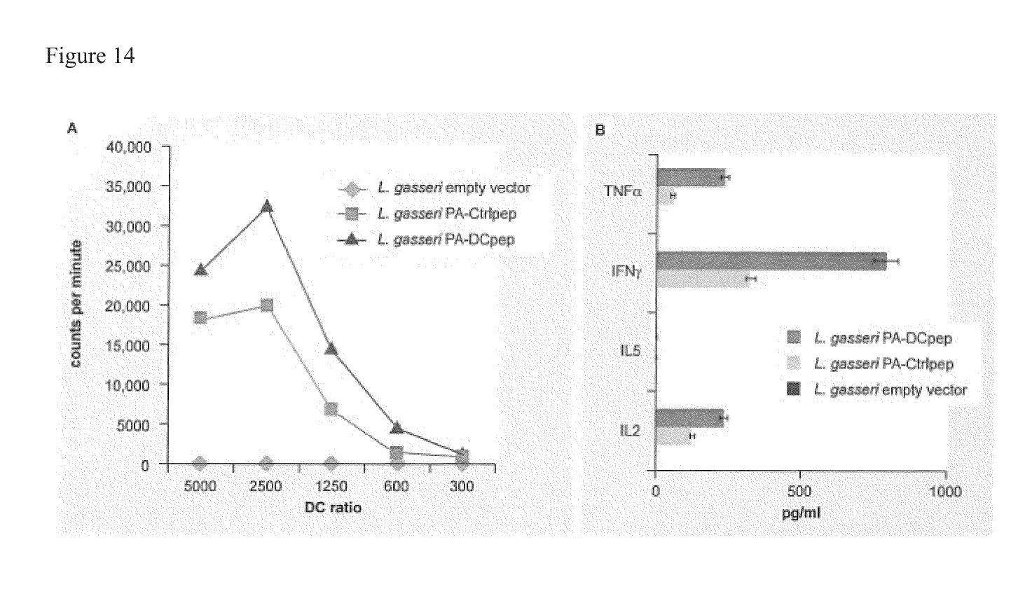

T cell immune responses. T cell immune responses against anthrax Sterne infection were determined using mesenteric LNs derived from the groups of mice that survived anthrax Sterne challenge. Data show that T cells derived from the mice that were vaccinated with L. gasseri expressing PA-DCpep fusion protein induced better proliferative and PA-specific T cell recall immune responses by producing higher levels of IFNγ, TNFα and IL-2 cytokines when compared with T cells derived from mice that were vaccinated with PA-Ctrlpep expressing L. gasseri (FIGS. 14A&B). These data indicate that L. gasseri expressing the PA-DCpep fusion skewed T cells towards Th1 polarization as demonstrated previously (Glomski et al., J. Immunol. 172:7425 (2007)). Such T cell immune responses differ significantly from CD4₊ T cell polarization derived from an AVA-vaccinated cohort. This effect is due to aluminum hydroxide gel acting as adjuvant that induces Th2 responses (Kwok et al., Infect. Immunol. 76:4538 (2008)).

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the following claims.

What is claimed is:

1. A composition comprising a nucleic acid encoding a fusion polypeptide comprising a dendritic cell-targeting peptide fused to an antigen of interest.

2. The composition of claim 1, wherein said dendritic cell-targeting peptide comprises the sequence FYPSYHSTPQRP.

3. A probiotic lactic acid bacteria comprising the composition of claim 1.

4. The bacteria of claim 3, wherein said bacteria is selected from the group consisting of Lactobacillus, Leuconostoc, Pediococcus, Lactococcus, and Streptococcus

5. The bacteria of claim 4, wherein said bacteria is selected from the group consisting of Lactobacillus gasseri and Lactobacillus acidophilus.

6. The composition of claim 1, wherein said antigen of interest is selected from the group consisting of a viral antigen and a bacterial antigen.

7. An immunization method, comprising
administering a composition comprising a bacteria comprising a nucleic acid encoding a fusion polypeptide comprising a dendritic cell-targeting peptide fused to an antigen of interest to a subject,
wherein said administering confers immunity to said antigen to said subject.

8. The method of claim 7, wherein said administration is oral

9. The method of claim 7, wherein said administration is intranasal.

10. The method of claim 7, wherein said dendritic cell-targeting peptide comprises the sequence FYPSYHSTPQRP.

11. The method of claim 7, wherein said bacteria is a probiotic lactic acid bacteria.

12. The method of claim 11, wherein said bacteria is selected from the group consisting of Lactobacillus, Leuconostoc, Pediococcus, Lactococcus, and Streptococcus

13. The method of claim 12, wherein said bacteria is selected from the group consisting of Lactobacillus gasseri and Lactobacillus acidophilus.


SEQUENCE LISTING (63 sequences)

SEQ ID NO: 1 - protein, 12 aa, Artificial Sequence
  Synthetic
FYPSYHSTPQRP

SEQ ID NO: 2 - protein, 12 aa, Artificial Sequence
  Synthetic
EPIHPETTFTNN

SEQ ID NO: 3 - DNA, 30 bp, Artificial Sequence
  Synthetic
atgcggatcccaaaaaggagaacgtatatg

SEQ ID NO: 4 - DNA, 20 bp, Artificial Sequence
  Synthetic
gcaattaaccctcactaaag

SEQ ID NO: 5 - protein, 10 aa, Artificial Sequence
  Synthetic
PDSLRDLSVF

SEQ ID NO: 6 - protein, 9 aa, Artificial Sequence
  Synthetic
PYNYLSTEV

SEQ ID NO: 7 - protein, 10 aa, Artificial Sequence
  Synthetic
LFRNPHQALL

SEQ ID NO: 8 - protein, 10 aa, Artificial Sequence
  Synthetic
PGPTQCVNCS

SEQ ID NO: 9 - protein, 9 aa, Artificial Sequence
  Synthetic
PNQAQMRIL

SEQ ID NO: 10 - protein, 10 aa, Artificial Sequence
  Synthetic
GSGAFGTVYK

SEQ ID NO: 11 - protein, 9 aa, Artificial Sequence
  Synthetic
AFGTVYKGI

SEQ ID NO: 12 - protein, 9 aa, Artificial Sequence
  Synthetic
PYVSRLLGI

SEQ ID NO: 13 - protein, 10 aa, Artificial Sequence
  Synthetic
LQRYSEDPTL

SEQ ID NO: 14 - protein, 15 aa, Artificial Sequence
  Synthetic
YVQASGRVTVSTRRS

SEQ ID NO: 15 - protein, 15 aa, Artificial Sequence
  Synthetic
INSNGNLIAPRGYFK

SEQ ID NO: 16 - protein, 15 aa, Artificial Sequence
  Synthetic
MRTGKSSIMRSDAPI

SEQ ID NO: 17 - protein, 15 aa, Artificial Sequence
  Synthetic
CPKYVKQNTLKLATG

SEQ ID NO: 18 - protein, 15 aa, Artificial Sequence
  Synthetic
KQNTLKLATGMRNVP

SEQ ID NO: 19 - protein, 15 aa, Artificial Sequence
  Synthetic
AELLVALENQHTIDL

SEQ ID NO: 20 - protein, 15 aa, Artificial Sequence
  Synthetic
ALENQHTIDLTDSEM

SEQ ID NO: 21 - protein, 10 aa, Artificial Sequence
  Synthetic
KEIGNGCFEF

SEQ ID NO: 22 - protein, 10 aa, Artificial Sequence
  Synthetic
SGPDNGAVAV

SEQ ID NO: 23 - protein, 9 aa, Artificial Sequence
  Synthetic
YRYGNGVWI

SEQ ID NO: 24 - protein, 8 aa, Artificial Sequence
  Synthetic
SSISFCGV

SEQ ID NO: 25 - protein, 15 aa, Artificial Sequence
  Synthetic
MMIWHSNLNDATYQR

SEQ ID NO: 26 - protein, 15 aa, Artificial Sequence
  Synthetic
TRALVRTGMDPRMCS

SEQ ID NO: 27 - protein, 15 aa, Artificial Sequence
  Synthetic
PRMCSLMQGSTLPRR

SEQ ID NO: 28 - protein, 15 aa, Artificial Sequence
  Synthetic
MIKRGINDRNFWRGE

SEQ ID NO: 29 - protein, 15 aa, Artificial Sequence
  Synthetic
INDRNFWRGENGRKT

SEQ ID NO: 30 - protein, 15 aa, Artificial Sequence
  Synthetic
FWRGENGRKTRIAYE

SEQ ID NO: 31 - protein, 15 aa, Artificial Sequence
  Synthetic
NGRKTRIAYERMCNI

SEQ ID NO: 32 - protein, 15 aa, Artificial Sequence
  Synthetic
RIAYERMCNILKGKF

SEQ ID NO: 33 - protein, 15 aa, Artificial Sequence
  Synthetic
QVYSLIRPNENPAHK

SEQ ID NO: 34 - protein, 15 aa, Artificial Sequence
  Synthetic
IRPNENPAHKSQLVW

SEQ ID NO: 35 - protein, 9 aa, Artificial Sequence
  Synthetic
ASNENMETM

SEQ ID NO: 36 - protein, 8 aa, Artificial Sequence
  Synthetic
GGLPFSLL

SEQ ID NO: 37 - protein, 8 aa, Artificial Sequence
  Synthetic
RTFSFQLI

SEQ ID NO: 38 - protein, 8 aa, Artificial Sequence
  Synthetic
FSVIFDRL

SEQ ID NO: 39 - protein, 15 aa, Artificial Sequence
  Synthetic
CSQRSKFLLMDALKL

SEQ ID NO: 40 - protein, 15 aa, Artificial Sequence
  Synthetic
GWKEPNVVKPHEKGI

SEQ ID NO: 41 - protein, 10 aa, Artificial Sequence
  Synthetic
SSLENFRAYV

SEQ ID NO: 42 - protein, 8 aa, Artificial Sequence
  Synthetic
NGYIEGKL

SEQ ID NO: 43 - protein, 8 aa, Artificial Sequence
  Synthetic
GIPLYDAI

SEQ ID NO: 44 - protein, 15 aa, Artificial Sequence
  Synthetic
VSPLAVTWWNRNGPM

SEQ ID NO: 45 - protein, 15 aa, Artificial Sequence
  Synthetic
TNTVHYPKIYKTYFE

SEQ ID NO: 46 - protein, 15 aa, Artificial Sequence
  Synthetic
CKISPLMVAYMLERE

SEQ ID NO: 47 - protein, 8 aa, Artificial Sequence
  Synthetic
RSYLIRAL

SEQ ID NO: 48 - protein, 8 aa, Artificial Sequence
  Synthetic
GYEEFTMV

SEQ ID NO: 49 - protein, 8 aa, Artificial Sequence
  Synthetic
VLRGFLIL

SEQ ID NO: 50 - protein, 12 aa, Artificial Sequence
  Synthetic
XYPSYHSTPQRP

SEQ ID NO: 51 - protein, 12 aa, Artificial Sequence
  Synthetic
FXPSYHSTPQRP

SEQ ID NO: 52 - protein, 12 aa, Artificial Sequence
  Synthetic
FYXSYHSTPQRP

SEQ ID NO: 53 - protein, 12 aa, Artificial Sequence
  Synthetic
FYPXYHSTPQRP

SEQ ID NO: 54 - protein, 12 aa, Artificial Sequence
  Synthetic
FYPSXHSTPQRP

SEQ ID NO: 55 - protein, 12 aa, Artificial Sequence
  Synthetic
FYPSYXSTPQRP

SEQ ID NO: 56 - protein, 12 aa, Artificial Sequence
  Synthetic
FYPSYHXTPQRP

SEQ ID NO: 57 - protein, 12 aa, Artificial Sequence
  Synthetic
FYPSYHSXPQRP

SEQ ID NO: 58 - protein, 12 aa, Artificial Sequence
  Synthetic
FYPSYHSTXQRP

SEQ ID NO: 59 - protein, 12 aa, Artificial Sequence
  Synthetic
FYPSYHSTPXRP

SEQ ID NO: 60 - protein, 12 aa, Artificial Sequence
  Synthetic
FYPSYHSTPQXP

SEQ ID NO: 61 - protein, 12 aa, Artificial Sequence
  Synthetic
FYPSYHSTPQRX

SEQ ID NO: 62 - DNA, 20 bp, Artificial Sequence
  Synthetic
attaggtgcaagtatttgac

SEQ ID NO: 63 - DNA, 20 bp, Artificial Sequence
  Synthetic
aataccgctgatacagcaag